(19) United States
(12) Patent Application Publication
(10) Pub. No.: US 2007/0161546 A1
(43) Pub. Date: Jul. 12, 2007

(54) Methods and compositions for treatment of cancer

(76) Inventor: Colm King, Delray Beach, FL (US)

Correspondence Address: LAW OFFICES OF KHALILIAN SIRA, LLC, 9100 PERSIMMON TREE ROAD, POTOMAC, MD, 20854, US

(21) Appl. No.: 11/636,843

(22) Filed: Dec. 11, 2006

Related U.S. Application Data
(60) Provisional application No. 60/750,574, filed on Dec. 15, 2005.

(51) Int. Cl. A61K 38/17

(52) U.S. Cl. 514/8

(57) ABSTRACT

A novel method of treating and preventing cancer diseases is provided. In particular, the present invention relates to compositions and methods for inhibition of cancer and the diseases associated with such cancers. More particularly, the present invention relates to the inhibitory compounds comprising naturally occurring and man-made compositions comprising a substance exhibiting Tubercin and/or SSM activity and/or Z-100 or a functional derivative thereof.

### CROSS REFERENCE TO RELATED APPLICATIONS

This application claims benefit under 37 U.S.C. § 119(e) based on U.S. Provisional Application No. 60/750,574, filed Dec. 12, 2005, the entire content of which is incorporated herein by reference.

### FIELD OF THE INVENTION

The present invention relates to compositions and methods for inhibition of cancer and to therapeutic treatment of diseases or disorders caused by such cancers. More particularly, the present invention also relates to cancer inhibitory compounds comprising naturally occurring and man-made Tubercin, Specific Substance of Maruyama materials and/or Z-100 or a functional derivative thereof.

### BACKGROUND OF THE INVENTION

Cancer

Cancer is a general term frequently used to indicate any of the various types of malignant neoplasms (i.e., abnormal tissue that grows by cellular proliferation more rapidly than normal), most of which invade surrounding tissue, may metastasize to several sites, are likely to recur after attempted removal, and causes death unless adequately treated. Stedman's Medical Dictionary, Williams & Wilkins, Baltimore, Md., 26th ed. 1995. Although a variety of approaches to cancer therapy (e.g., surgical resection, radiotherapy, and chemotherapy) have been available and commonly used for many years, cancer remains one of the leading causes of death in the world.

Lung cancer incidence rates in men have reached an apparent plateau, but prostate cancer has increased dramatically. As a result, cancer of the prostate gland has become the most common type of cancer among both black (incidence rate of 163.1 per 100,000) and white (121.2 per 100,000) males. Lung cancer and colorectal cancer rates are the second and third highest, respectively, for both black and white males.

Breast cancer is by far the most common cancer among both white and black females. It occurs more frequently among white females (113.2 per 100,000) than among black females (94.0 per 100,000). Lung cancer and colorectal cancer are the second and third highest cancers, respectively, among white females compared to ranks of third and second highest, respectively, for black females. Even though lung and colorectal cancers are two of the most common cancers among females, their incidence is much lower than that for males. The fourth most common cancer for both white and black females is uterine cancer.

In general, tumors may be benign or malignant. Benign tumors are not cancerous. They can often be surgically removed and usually do not reoccur. Cells from benign tumors do not spread to other parts of the body. Most importantly, benign tumors are rarely life-threatening. Malignant tumors, on the other hand, are cancerous. Cells in malignant tumors are abnormal and divide without control or order. They are often invasive and damage adjacent tissues and organs. Also, cancer cells can metastasize from a malignant tumor and enter the bloodstream or the lymphatic system. That is the most common way cancer spreads from the original cancer site to form new tumors in other organs. Leukemia and lymphoma are cancers that arise in blood-forming cells. The abnormal cells circulate in the bloodstream and lymphatic system. They may also invade or infiltrate body organs and form tumors.

The hallmark of cancer is a capacity for unlimited, autonomous cellular proliferation. The discovery of a means of selective inhibition of proliferation in cancer cells without concomitant damage to the proliferative capacity of normal cells, could potentially provide new ways to halt the growth of tumours irrespective of their degree of differentiation, invasion or metastasis. It is interesting to understand how cancer cells divide and whether this may differ from normal cell division. Most treatments for cancer are broadly cytotoxic, generally targeting proliferating cells. Normal tissues also proliferate, however, and so are also damaged by cytotoxic agents. Identification of cancer specific markers that permit targeting or that provide specific targets for drug development would allow the development of treatments that are more specifically toxic to tumor tissues thus reducing the debilitating effects of chemotherapy.

No single drug or drug combination is curative for advanced metastatic cancer and patients typically succumb to the cancers in several years. Thus, new drugs or combinations that can prolong onset of life-threatening tumors and/or improve quality of life by further reducing tumor-load are very important.

Because of some of the difficulties and inadequacies of conventional therapy for cancer, new therapeutic modalities are therefore still desirable. Accordingly, despite some successful therapies using a variety of anti-cancer agents additional and improved treatments directed against various cancer targets are still desperately needed.

This invention addresses a long-felt need for safe and effective compositions and methods of treatment of cancer.

### SUMMARY OF THE INVENTION

The present invention relates to therapeutically active compounds, pharmaceutical formulations containing said compounds and the use of said compounds in treatment and prophylaxis of cancer and related diseases and/or indications.

In one embodiment, the therapeutically active compound that inhibits cancer or tumor development in a mammal comprises Tubercin, Tubercin-3, Tubercin-5, Tubercin-7, SSM (otherwise known as SSMA or Specific Substance of Maruyama), or Z-100 or any combination thereof.

In yet another embodiment, the agent that inhibits cancer or tumor development in a mammal comprises a Tubercin, Tubercin-3, Tubercin-5, Tubercin-7, SSM, or Z-100-based oligosaccharide-protein conjugate or lipid arabinomannan-protein conjugate or any combination thereof.

The agent that inhibits cancer or tumor development in a mammal can include, but is not limited to, small organic molecules including naturally-occurring, synthetic, and biosynthetic molecules, small inorganic molecules including naturally-occurring and/or synthetic molecules.

One aspect of the present invention is to provide clinically acceptable cancer inhibitors exhibiting relatively high anti-cancer activity at relatively low concentrations.

The invention further provides pharmaceutical compositions comprising such agents.

The present invention provides methods for treating cancer in a mammal comprising administering to a subject in need thereof a therapeutically effective amount of a composition comprising a substance exhibiting Tubercin and/or SSM activity and/or Z-100 activity or a finctional derivative thereof; and a pharmaceutically acceptable excipient.

Also provided is a method of inhibiting a cancer of a mammal, which comprises administering to a mammal susceptible to a cancer an effective amount of a substance exhibiting Tubercin and/or SSM activity and/or Z-100 activity or a functional derivative thereof; and a pharmaceutically acceptable excipient.

In another aspect, the present invention further provides a method of controlling cell proliferation in cancer cells within a warm-blooded animal including humans afflicted with cancer comprising administering to the warm-blooded animal an antiproliferative effective amount of a substance exhibiting Tubercin and/or SSM activity and/or Z-100 activity or a finctional derivative thereof or the pharmaceutically composition of the present invention. As used herein an “antiproliferative effective amount” is defined as the amount sufficient to bring about the slowing, halting or preventing of the onset of cell reproduction or proliferation in cancer cells treated in the warm-blooded animal. The precise amount that is considered effective for a particular therapeutic purpose will, of course, depend upon the specific circumstance of the warm-blooded animal being treated and the magnitude of the effect desired.

In another aspect, the present invention further provides a method of inhibiting metastasis in a warm-blooded animal including humans afflicted with cancer comprising administering to the warm-blooded animal a metastasis-inhibiting effective amount of a substance exhibiting Tubercin and/or SSM activity and/or Z-100 activity or a functional derivative thereof or the pharmaceutically composition of the present invention. As used herein a “metastasis-inhibiting effective amount” is defined as the amount sufficient to bring about the slowing, halting or preventing of the onset of metastasis by the cancer being treated in the warm-blooded animal. The precise amount that is considered effective for a particular therapeutic purpose will, of course, depend upon the specific circumstance of the warm-blooded animal being treated and the magnitude of the effect desired.

In another aspect, the present invention further provides a method of inhibiting protein degradation such as caused by metastasizing cancer cells in a warm-blooded animal including humans afflicted with cancer comprising administering to the warm-blooded animal a protein degradation-inhibiting effective amount of a substance exhibiting Tubercin and/or SSM activity and/or Z-100 activity or a functional derivative thereof or the pharmaceutical composition of the present invention. As used herein the term “protein degradation” shall refer to the degradation of all types of proteins including all proteins found in the extracellular matrix of tissues of warm-blooded animals. A common example of such proteins is collagen. As used herein a “protein degradation-inhibiting effective amount” is defined as the amount sufficient to bring about the slowing, halting or preventing of the onset of protein degradation such as that caused by the cancer being treated in the warm-blooded animal. The precise amount that is considered effective for a particular therapeutic purpose will, of course, depend upon the specific circumstance of the warm-blooded animal being treated and the magnitude of the effect desired.

Without intending to be limited to any particular mechanism, it is postulated that Z-100 restores the balance of Th1/Th2 cell responses via the suppression of glucocorticoid-genesis by Z-100-induced IFN-gamma.

Without intending to be limited to any particular mechanism, it is postulated that SSM acts by serving as a biological response modifier that induces encapsulation of tumor growths, possibly preventing their spread and metastasis, and that SSM may benefit patients in whom tumor is inoperable and resistant to conventional chemotherapy.

In another aspect, the pharmaceutical combinations of the present invention are useful in cancer therapy, in particular in the treatment of cancers selected from the group comprising AIDS-Associated Cancers, Bladder Cancer, Bone Cancer, Brain & Spinal Cord Cancers, Metastatic Brain Tumors, Pediatric Brain Tumors, Breast Cancer, Male Breast Cancer, Cervical Cancer, Colorectal Cancer, Endometrial & Other Uterine Cancers, Esophageal Cancer, Gallbladder & Bile Duct Cancers, Gastric (Stomach) Cancer, Gestational Trophoblastic Disease, Head & Neck Cancers, Kidney Cancer, Leukemia, Liver Cancer, Liver Metastases, Lung Cancer, Lymphomas, Melanoma, Multiple Myeloma, Myelodysplastic Syndrome, Neuroblastoma, Ovarian Cancer, Pancreatic Cancer, Pediatric Cancers, Pituitary Tumors, Prostate Cancer, Rare Hematologic Disorders,.Rare Solid Tumors, Retinoblastoma, Skin Cancer, Soft-Tissue Sarcoma, Testicular Cancer, Thyroid Cancer, Uterine Cancers, Wilms' Tumor, Bronchus Cancer, Colon and Rectum Cancer, Urinary Cancer, Non-Hodgkin Lymphoma, Melanomas of the Skin, Kidney and Renal Cancer, Pelvis Cancer, Pancreatic Cancer, Oral Cavity Cancer and Pharynx Cancer, Ovary Cancer, Stomach Esophagus Cancer, Intrahepatic Bile Duct Cancer, Cervix Cancer, Larynx Cancer, Acute Myeloid Leukemia, Chronic Lymphocytic Leukemia, Soft Tissue Cancer including Heart, GIST (Gastro-Intestinal Stromal Tumors), Small Intestine Cancer, Chronic Myeloid Leukemia, Acute Lymphocytic Leukemia Anus, Anal, Canal and Anorectum Cancers, Vulva Cancer, Gallbladder Cancer, Pleura Cancer, Malignant Mesothelioma, Cancer of the Bones and Joints, Hypopharynx Cancer, Eye and Orbit Cancer, Nose and Nasal Cavity Cancer, Middle Ear Cancer, Nasopharynx Cancer, Ureter Cancer, Peritoneum Cancer, Omentum and Mesentery Cancer, and Gastrointestinal Carcinoid Tumors or any combination thereof.

In yet another aspect, the pharmaceutical combinations of the present invention are useful in cancer therapy, in particular in the treatment of cancers selected from the group comprising colorectal cancer, gastric cancer, ovarian cancer, osteosarcoma, hepatocellular carcinoma, Burkitt's lymphoma, primary effusion lymphomas, angioimmunoblastic lymphadenopathy, acquired immune deficiency syndrome (AIDS)-related lymphoma, T-cell lymphomas, oral hairy leukoplakia, lymphoproliferative disease, lymphoepithelial carcinoma, body-cavity-based lymphoma or B-cell lymphomas, non-keratinising carcinoma, squamous cell nasopharyngeal carcinoma, kidney transplant-associated epithelial tumors, angiosarcoma, Kaposi's sarcoma, angiolymphoid hyperplasia, prostatic neoplasm, retinoblastoma, Li-Fraumeni syndrome, Gardner's syndrome, Werner's syndrome, nervoid basal cell carcinoma syndrome, neurofibromatosis type 1, cervical dysplasia, neuro-blastoma, primary macroglobulinemia, insulinoma, mycosis fungoides, osteogenic sarcoma, premalignant skin lesions (topical), rhabdomyosarcoma, osteogenic, polycythemia vera, essential thrombocytosis or any combination thereof.

In another aspect, compounds of the present invention may additionally be combined with chemotherapeutic agents and/or potentiators to provide a combination therapy for any of methods for treating any of the aforementioned cancers. Combination therapy is intended to include any chemically compatible combination of a substance exhibiting Tubercin and/or SSM activity and/or Z-100 activity or a functional derivative thereof of the present invention with other compounds of the present invention or other compounds outside of the present invention, as long as the combination does not eliminate the activity of the substance exhibiting Tubercin and/or SSM activity and/or Z-100 activity or a functional derivative thereof of the present invention. For example, one or more compounds may be combined with a potentiator or with a chemotherapeutic agent. The active agent can be coadministered, for example, in the form of a tablet or capsule, liposome, as an agglomerated powder, or in a liquid form. The amount of chemotherapeutic agent or potentiator used can be lower than that of the substance exhibiting Tubercin and/or SSM activity and/or Z-100 activity or a functional derivative thereof. It will be present in a dosage unit in an amount that provides an operative combination with the substance exhibiting Tubercin and/or SSM activity and/or Z-100 activity or a functional derivative thereof. The dosage of the chemotherapeutic agent or the potentiator can range from about 0.5 mg/kg body weight to about 400 mg/kg body weight.

Combination therapy can be sequential, that is the treatment with one agent first and then the second agent, or it can be treatment with both agents at the same time. The sequential therapy can be within a reasonable time after the completion of the first therapy before beginning the second therapy. The treatment with both agents at the same time can be in the same daily dose or in separate doses. For example, treatment with one agent on day 1 and the other on day 2. The exact regimen will depend on the disorder being treated, the severity of the disorder, and the response to the treatment.

Thus, in another aspect, there is provided a method of treating a patient having cancer, in particular in the treatment of cancer selected from the group comprising lung cancer, prostate cancer, bladder cancer, colorectal cancer, pancreatic cancer, gastric cancer, breast cancer, ovarian cancer, soft tissue sarcoma, osteosarcoma, hepatocellular carcinoma, leukemia and lymphomas, Burkitt's lymphoma, primary effusion lymphomas, multiple myeloma, angioimmunoblastic lymphadenopathy, acquired immune deficiency syndrome (AIDS)-related lymphoma, T-cell lymphomas, oral hairy leukoplakia, lymphoproliferative disease, lymphoepithelial carcinoma, body-cavity-based lymphoma or B-cell lymphomas, non-keratinising carcinoma, squamous cell nasopharyngeal carcinoma, kidney transplant-associated epithelial tumors, malignant mesothelioma, angiosarcoma, Kaposi's sarcoma, angiolymphoid hyperplasia, prostatic neoplasm, cervical cancer, neoplasms of the vulva, retinoblastoma, Li-Fraumeni syndrome, Gardner's syndrome, Werner's syndrome, nervoid basal cell carcinoma syndrome, neurofibromatosis type 1, cervical dysplasia, Hodgkin's disease, non-Hodgkin's lymphomas, Acute and chronic lymphocytic leukemias, multiple myeloma, neuro-blastoma, breast, ovary, lung, Wilms' tumor, cervix, testis, soft-tissue sarcomas, Chronic lymphocytic leukemia, primary macroglobulinemia, Prostate, Bladder, Chronic granulocytic leukemia, primary brain tumors, malignant melanoma, small-cell lung, stomach, colon, Malignant pancreatic, insulinoma, malignant carcinoid, chorio-carcinoma, mycosis fungoides, head and neck, lung, osteogenic sarcoma, pancreas, urinary, bladder, premalignant skin lesions (topical), Hairy cell leukemia, testis, Kaposi's sarcoma, Choriocarcinoma, rhabdomyosarcoma, testis Kaposi's sarcoma Soft-tissue, osteogenic, genitourinary tract, thyroid, neuroblastoma hypercalcemia, cervix, Acute lymphocytic leukemia, melanoma, carcinoid, renal cell, endometrium, polycythemia vera, essential thrombocytosis, Adrenal cortex, Estrogen-receptor-positive breast, or any combination thereof comprising administering to said patient a therapeutically effective amount of a substance exhibiting Tubercin and/or SSM activity and/or Z-100 activity or a functional derivative thereof and at least one further therapeutic agent. In certain embodiments, the at least one further therapeutic agent is the agent or agents specifically listed for each specific cancer in Table 1.

In yet another embodiment, with respect to the use of the compositions and methods of the present invention to prevent, ameliorate or treat cancer, specifically excluded within the scope of the present invention is the use of Tubercin-3 for treating terminal-cancer patients as disclosed in Chung, T. H., J. Korean Med. Ass., 17, 427-431(1974); Chung, T. H. et al., Yonsei Med. J., 17, 131-135(1976).

In yet another embodiment, with respect to the use of the compositions and methods of the present invention to prevent, ameliorate or treat cancer, specifically excluded within the scope of the present invention is the use of SSMA or Z-100 to treat cancers selected from the group consisting of ovarian cancer, primary uterine cervix cancer, melanoma or any combination thereof.

In yet another embodiment, with respect to the use of the compositions and methods of the present invention to prevent, ameliorate or treat cancer, specifically excluded within the scope of the present invention is the use of isolated components of an attenuated strain of Mycobacterium bovis, Bacillus Calmette-Guerin (BCG vaccine) to treat cancers selected from the group consisting of superficial bladder cancer, melanoma, lung cancer and leukemia or any combination thereof as disclosed in U.S. Pat. No. 5,712,123.

In one embodiment, the reduction or inhibition of pain, inhibition of cancer or tumor development, inhibition of metastasis, inhibition of protein degradation, inhibition of cell proliferation in cancer cells, and/or inhibition of symptoms associated with one or more of each of the above-recited cancer diseases and/or indications is on the order of about 10-20% reduction or inhibition. In another embodiment, the reduction or inhibition of cancer or tumor development, inhibition of metastasis, inhibition of protein degradation, inhibition of cell proliferation in cancer cells, and/or symptoms is on the order of 30-40%. In another embodiment, the reduction or inhibition of cancer or tumor development, inhibition of metastasis, inhibition of protein degradation, inhibition of cell proliferation in cancer cells, and/or inhibition of symptoms is on the order of 50-60%. In yet another embodiment, the reduction or inhibition of the inhibition of cancer or tumor development, inhibition of metastasis, inhibition of protein degradation, inhibition of cell proliferation in cancer cells, and/or inhibition of symptoms associated with each of the recited cancer diseases and/or indications is on the order of 75-100%. It is intended herein that the ranges recited also include all those specific percentage amounts between the recited ranges. For example, the range of about 75 to 100% also encompasses 76 to 99%, 77 to 98%, etc, without actually reciting each specific range therewith.

It is thus another aspect of the present invention to provide a novel method for treating an AIDS-related cancer which comprises administering to a host in need thereof a therapeutically effective combination of (a) a substance exhibiting Tubercin and/or SSM activity and/or Z-100 activity or a functional derivative thereof and (b) one or more compounds selected from the group consisting of retroviral reverse transcriptase inhibitors, retroviral protease inhibitors, or an entry inhibitor. Accordingly, reverse transcriptase inhibitor can be selected from a group including nucleoside RT inhibitors: Retrovir (AZT/zidovudine; Glaxo Wellcome); Combivir (Glaxo Wellcome); Epivir (3TC, lamivudine; Glaxo Wellcome); Videx (ddI/didanosine; Bristol-Myers Squibb); Hivid (ddC/zalcitabine; Hoffmann-La Roche); Zerit (d4T/stavudine; Bristol-Myers Squibb); Ziagen (abacavir, 1 592U89; Glaxo Wellcome); tenofovir, emtricitabine, Hydrea (Hydroxyurea/HO; nucleoside RT potentiator from Bristol-Myers Squibb) or Non-nucleoside reverse transcriptase inhibitors (NNRTIs): Viramune (nevirapine; Roxane Laboratories); Rescriptor (delavirdine; Pharmacia & Upjohn); Sustiva (efavirenz, DMP-266; DuPont Merck); Preveon (adefovir dipivoxil, bis-POM PMEA; Gilead). Protease inhibitors (PI's) are selected from Fortovase (saquinavir; Hoffmann-La Roche); Norvir (ritonavir; Abbott Laboratories); Crixivan (indinavir; Merck & Company); Viracept (nelfinavir; Agouron Pharmaceuticals); Angenerase (amprenavir/141W94; Glaxo Wellcome), atazanavir, Kaletra (lopinavir/ritonavir) VX-478, KNI-272, CGP-61755, and U-103017, or the entry inhibitor T20 (Fuzeon or enfuvirtide), or any combination thereof.

For each of the above-recited methods of the present invention, the therapeutically effective amount of the one or more substances exhibiting Tubercin and/or SSM activity and/or Z-100 activity or a functional derivative thereof and a pharmaceutically acceptable excipient thereof may be administered to a subject in need thereof in conjunction with a therapeutically effective amount of one or more anti-viral drugs and/or inflammatory compounds and/or a therapeutically effective amount of one or more immunomodulatory agents.

In certain embodiments of the methods of the present invention, the anti-inflammatory compound or immunomodulatory drug comprises interferon; interferon derivatives comprising betaseron, .beta.-interferon; prostane derivatives comprising iloprost, cicaprost; glucocorticoids comprising cortisol, prednisolone, methyl-prednisolone, dexamethasone; immunsuppressives comprising cyclosporine A, FK-506, methoxsalene, thalidomide, sulfasalazine, azathioprine, methotrexate; lipoxygenase inhibitors comprising zileutone, MK-886, WY-50295, SC-45662, SC-41661A, BI-L-357; leukotriene antagonists; peptide derivatives comprising ACTH and analogs thereof; soluble TNF-receptors; TNF-antibodies; soluble receptors of interleukins, other cytokines, T-cell-proteins; antibodies against receptors of interleukins, other cytokines, T-cell-proteins; and calcipotriols and analogues thereof taken either alone or in any combination thereof.

In yet another aspect, the present invention is directed to a method of relieving or ameliorating cancer or tumor development, metastasis, protein degradation, cell proliferation in cancer cells, and/or inhibition of symptoms associated with any one or more of the above-identified cancer diseases and/or cancer indications in a mammal suffering from any one or more of the above-identified cancer diseases or cancer indications which comprises administering to the mammal in need thereof a therapeutically effective pain or symptom-reducing amount of a pharmaceutical composition comprising effective amounts of a substance exhibiting Tubercin and/or SSM activity and/or Z-100 activity or a functional derivative thereof and a pharmaceutically acceptable excipient, either alone or in combination with one or more anti-inflammatory compounds or immunomodulatory agents, wherein said substance exhibiting Tubercin and/or SSM activity and/or Z-100 activity or a functional derivative thereof is sufficient to inhibit the cancer or tumor development, metastasis, protein degradation, and/or cell proliferation in cancer cells.

The present invention also relates to the combined use of the pharmaceutical composition comprising a substance exhibiting Tubercin and/or SSM activity and/or Z-100 activity or a functional derivative thereof and a pharmaceutically acceptable excipient in combination with one or more antibacterial or antiviral compositions or any combination thereof for treating any one of the aforementioned cancer diseases and/or indications, or any combination thereof.

The present invention thus provides methods for therapeutically or prophylactically treating cancer and/or cancer indications in a subject.

In one embodiment, the method for therapeutically treating cancer comprises the step of administering pharmaceutically effective amounts of a substance exhibiting Tubercin and/or SSM activity and/or Z-100 activity or a functional derivative thereof and a pharmaceutically acceptable excipient to the subject after development of the cancer disease and/or cancer indication.

In another embodiment, the method for prophylactically treating cancer comprises the step of administering pharmaceutically effective amounts of a substance exhibiting Tubercin and/or SSM activity and/or Z-00 activity or a functional derivative thereof and a pharmaceutically acceptable excipient to the subject prior to the occurrence of the cancer disease and/or cancer indication.

Either methodology inhibits cancer or tumor development, metastasis, protein degradation, and/or cell proliferation associated with cancer in a mammal.

The preferred doses for administration can be anywhere in a range between about 10 ng and about 10 mg per ml or mg of the formulation. The therapeutically effective amount of a substance exhibiting Tubercin and/or SSM activity and/or Z-100 activity or a functional derivative thereof can be also measured in molar concentrations and may range between about 1 nM and about 10 mM. The formulation is also contemplated in combination with a pharmaceutically or cosmetically acceptable carrier. The precise doses can be established by well known routine clinical trials without undue experimentation.

In yet another aspect, the present invention provides a method for preventing a symptom of a given cancer in a subject thought to be at risk for developing a given cancer comprising administering to the subject a pharmaceutically effective amount of a substance exhibiting Tubercin and/or SSM activity and/or Z-100 activity or a functional derivative thereof, wherein said substance exhibiting Tubercin and/or SSM activity and/or Z-100 activity or a functional derivative thereof substance inhibits the cancer or tumor development, metastasis, protein degradation, and/or cell proliferation associated with such cancer, and wherein if the subject contracts cancer, a symptom of said cancer is prevented.

In another aspect, the present invention provides a method for preventing a symptom of a given cancer in a subject suspected of having been exposed to a given cancer comprising administering to the subject a pharmaceutically effective amount of a substance exhibiting Tubercin and/or SSM activity and/or Z-100 activity or a functional derivative thereof, wherein said substance exhibiting Tubercin and/or SSM activity and/or Z-100 activity or a functional derivative thereof inhibits the cancer or tumor development, metastasis, protein degradation, and/or cell proliferation associated with such cancer, and wherein if the subject contracts cancer, a symptom of said cancer is prevented.

In another aspect, the present invention provides a method for ameliorating a symptom of a given cancer in a subject in need of said amelioration comprising administering to the subject a pharmaceutically effective amount of a substance exhibiting Tubercin and/or SSM activity and/or Z-100 activity or a functional derivative thereof, wherein said substance exhibiting Tubercin and/or SSM activity and/or Z-100 activity or a functional derivative thereof inhibits the cancer or tumor development, metastasis, protein degradation, and/or cell proliferation associated with such cancer, and wherein if the subject contracts cancer, a symptom of said cancer is ameliorated.

Among preferred compounds to treat such cancers are substantially purified natural or synthetic Tubercin and/or SSM compounds and/or Z-100 compounds or functional derivatives thereof. Tubercin and/or SSM and similarly active compounds may be identified by a series of assays wherein a compound (natural or synthetic Tubercin and/or SSM and/or Z-100 or a functional derivative thereof) will exhibit cancer inhibitory activity versus control in an assay. For example, and not by way of limitation, one of these assays comprises the murine S180 sarcoma assay described in Example 1 herein. Other similar cancer inhibitory-based assays known to those of skill in the art may be used to identify natural or synthetic Tubercin and/or SSM compounds or finctional derivatives thereof for use in any one of the aforementioned methods of the present invention.

The treatment and prevention of virus-induced tumors by administering the substance exhibiting Tubercin and/or SSM activity or a functional derivative thereof is yet another aspect of this invention. Yet another preferred embodiment of this invention is to provide a substance exhibiting Tubercin and/or SSM activity and/or Z-100 activity or functional derivative thereof for treatment of various types of cancer that may or may not be virus-induced but are capable of metastasizing. Such tumors may comprise fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, rhabdosarcoma, colorectal carcinoma, pancreatic cancer, breast cancer, ovarian cancer, prostate cancer, melanoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, myeloma, lymphoma, and leukemia or any combination thereof

In yet another aspect of the present invention a method is provided for treating and/or preventing cancer in a mammal comprising administering Tubercin and/or SSM in combination with one or more proteins or peptides. In the aspect of this invention pertaining to the Tubercin and/or SSM and/or Z-100-protein/peptide combination therapy, among the preferred compounds to treat any one of the aforementioned cancers or cancer indications is a substantially purified natural or recombinant protein or peptide. Such proteins or peptide fragments thereof can be associated with one or more modified saccharides or modified oligosaccharides using chemical synthesis methods known to those of skill in the art so as to form a glycoprotein or glycopolypeptide. Representative examples of classes of proteins which can be used as the non-saccharide portion of a molecule include antibodies, enzymes, growth factors, cytokines and chemokines.

Antibodies which can be associated with a modified saccharide, as described herein, include CDP-571, gemtuzumab ozogamicin, biciromab, imciromab, capromab, .sup.111 lindium satumomab pendetide, bevacizumab, ibritumomab tiuxetan, cetuximab, sulesomab, afelimomab, HuMax-CD4, MDX-RA, palivizumab, basiliximab, inolimomab, lerdelimumab, pemtumomab, idiotypic vaccine (CEA), Titan, Leucotropin, etanercept, pexelizumab, alemtuzumab, natalizumab, efalizumab, trastuzumab, epratuzumab, palivizumab, daclizumab, lintuzumab, Cytogam, Engerix-B, Enbrel, Gamimune (IgG), Meningitec, Rituxan, Synagis, Reopro, Herceptin, Sandoglobulin, Menjugate, and BMS-188667. Growth factors, enzymes and receptors which can be used as non-saccharide moieties include Benefix, Meningitec, Refacto, Procit, Epogen, Eprex, Intron A, Neupogen, Humulin, Avonex, Betaseron, Cerezyme, Genotropin, Kogenate, NeoRecormon, Gonal-F, Humalog, NovoSeven, Puregon, Norditropin, Rebif, Nutropin, Activase, Espo, Neupogen, Integrilin, Roferon, Insuman, Serostim, Prolastin, Pulmozyme, Granocyte, Creon, Hetrodin HP, Dasen, Saizen, Leukine, Infergen, Retavase, Proleukin, Regranex, Z-100, somatropin, Humatrope, Nutropin Depot, somatropin, epoetin delta, Eutropin, ranpimase, infliximab, tifacogin, oprelvekin, interferon-alpha, aldesleukin, OP-1, drotrecogin alfa, tasonermin, oprelvekin, etanercept, afelimomab, daclizumab, thymosin alpha 1, becaplermin, and A-74187. Other non-saccharide moieties which can be used include pexelizumab, anakinra, darbepoetin alfa, insulin glargine, Avonex, alemtuzumab, Leucotropin, Betaseron, aldesleukin, domase alfa, tenecteplase, oprelvekin, choriogonadotropin alfa, and nasaruplase, or any combination thereof.

In the aspect of this invention pertaining to the Tubercin and/or SSM-conjugate therapy, the substance exhibiting Tubercin and/or SSM activity and/or Z-100 activity or a functional derivative thereof may also be directly conjugated to the compound or functional derivative thereof using either routine or advanced synthetic chemical reactions known to those of skill in the chemical art.

In each of the above-recited methods, the substance exhibiting Tubercin and/or SSM activity and/or Z-100 activity or a functional derivative thereof may be part of a fusion polypeptide, wherein said fusion polypeptide comprises a substance exhibiting Tubercin and/or SSM activity or a functional derivative thereof and an amino acid sequence heterologous to said substance exhibiting Tubercin and/or SSM activity or a functional derivative thereof.

In certain embodiments, the fusion polypeptide contemplated for use in the methods of the present invention comprises a human immunoglobin constant region, such as for example, a human IgGI constant region, including a modified human IgGl constant region wherein the IgGI constant region does not bind the Fc receptor and/or does not initiate antibody-dependent cellular cytotoxicity (ADCC) reactions.

In yet other embodiments, the fusion polypeptides contemplated for use in the methods of the present invention can additionally comprise an amino acid sequence that is useful for identifying, tracking or purifying the fusion polypeptide, e.g., the fusion polypeptide can further comprise a FLAG or HIS tag sequence. The fusion polypeptide can additionally further comprise a proteolytic cleavage site which can be used to remove the heterologous amino acid sequence from substance exhibiting Tubercin and/or SSM activity and/or Z-100 activity or a functional derivative thereof.

In yet another aspect of the present invention, for each of the above-recited methods, the one or more substances exhibiting Tubercin and/or SSM activity and/or Z-100 activity or a functional derivative thereof may themselves be administered as an adjuvant, wherein the therapeutically effective amount of the one or more substances exhibiting Tubercin and/or SSM activity and/or Z-100 activity or a functional derivative thereof acts as an immunostimulant or immunomodulator to be used in conjunction with one or more of the pharmaceutical drugs listed in the Physicians Desk Reference as described infra.

In each of the above-recited methods, the substance exhibiting Tubercin and/or SSM activity and/or Z-100 activity or a functional derivative that inhibits a cancer in a mammal can comprise small organic molecules or compounds including naturally-occurring, synthetic, and biosynthetic molecules or compounds, small inorganic molecules or compounds including naturally-occurring and/or synthetic molecules or compounds provided that said molecules or compounds specifically exhibit Tubercin and/or SSM activity and/or Z-100 activity or Tubercin and/or SSM-like activity and/or Z-100 like activity as can be tested by the in vitro assays described in detail infra.

While the invention has been illustrated using Tubercin, SSM and functional derivatives thereof for each of the above-mentioned methods, it should be readily apparent that each of the aforementioned methods may be practiced without undue experimentation using other bacterial cell wall extracts that have been previously used as immune stimulants and anti-tumor agents. Accordingly, representative examples of such bacterial cell wall extracts that may be used in each of the aforementioned methods are all those bacterial species from the genera Mycobacterium, Propionibacterium, Nocardia, and Actinomycetes, as well as Bacillus Calmette-Guerin (BCG), polysaccharide K, Beta 1,3-glucan, and extracts of Bifidobacterium, L. Lactis, L. fermentum, L. acidopholus, and S. lactis, as if specifically set forth herein in their entirety. For example, and not by way of limitation, muramyl peptidyl glycan complex (MPGC) is a non-toxic bacterial cell wall extract of Lactobacillus fermentum that contains muramic acid moieties attached to variable length mannose-rich polysaccharides. The mannose rich polysaccharides promote internalization of the entire muramic acid-containing complex and may be used in each of the methods of the present invention.

In one aspect of the invention, the pharmaceutical compositions of the present invention are administered orally, systemically, via an implant, intravenously, topically, intrathecally, intracranially, intraventricularly, by inhalation or nasally.

In certain embodiments of the methods of the present invention, the subject or mammal is a human.

In other embodiments of the methods of the present invention, the subject or mammal is a veterinary and/or a domesticated mammal.

There has been thus outlined, rather broadly, the important features of the invention in order that a detailed description thereof that follows can be better understood, and in order that the present contribution can be better appreciated. There are additional features of the invention that will be described hereinafter.

In this respect, before explaining at least one embodiment of the invention in detail, it is to be understood that the invention is not limited in its application to the details as set forth in the following description and figures. The present invention is capable of other embodiments and of being practiced and carried out in various ways. Additionally, it is to be understood that the terminology and phraseology employed herein are for the purpose of description and should not be regarded as limiting.

As such, those skilled in the art will appreciate that the conception, upon which this disclosure is based, can readily be used as a basis for designing other methods for carrying out the several features and advantages of the present invention. It is important, therefore, that the claims be regarded as including such equivalent constructions insofar as they do not depart from the spirit and scope of the present invention.

### DETAILED DESCRIPTION OF THE INVENTION

Standard Methods

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition 1989, Cold Spring Harbor Laboratory Press, Cold Spring Harbor, N.Y.; Animal Cell Culture, R. I. Freshney, ed., 1986).

Therapeutic Methods

The present invention provides methods for treating cancer comprising administering to a subject in need thereof of a therapeutically effective amount of a composition comprising an effective amount of a substance exhibiting Tubercin and/or SSM activity and/or Z-100 or a functional derivative thereof; and a pharmaceutically acceptable excipient.

Therefore, administration of a dosage of the invention composition, i.e., substance exhibiting Tubercin and/or SSM activity and/or Z-100 or a functional derivative thereof, can be beneficial for the treatment of cancer diseases or disorders. In a preferred aspect, the agent is an analog of a substance exhibiting Tubercin and/or SSM activity or a functional derivative thereof that can cross the blood brain barrier, which would allow for intravenous or oral administration. Many strategies are available for crossing the blood brain barrier, including but not limited to, increasing the hydrophobic nature of a molecule; introducing the molecule as a conjugate to a carrier, such as transferrin, targeted to a receptor in the blood brain barrier; and the like. In another embodiment, the agent can be administered intracranially or, more directly, intraventricularly. In yet another embodiment, the agent can be administered by way of inhalation or nasally.

In a further embodiment, the methods and compositions of the invention are useful in the therapeutic treatment of cancer diseases or disorders of the immune system. In a yet further embodiment, diseases can be prevented by the timely administration of the agent of the invention as a prophylactic, prior to onset of symptoms, or signs, or prior to onset of severe symptoms or signs of a cancer disease. Thus, a patient at risk for a particular cancer disease can be treated with a substance exhibiting Tubercin and/or SSM activity and/or Z-100 or a functional derivative thereof as a precautionary measure.

In yet another aspect, the present invention is directed to a method of relieving or ameliorating cancer or tumor development, metastasis, protein degradation, cell proliferation in cancer cells, and/or inhibition of symptoms associated with any one or more of the above-identified cancer diseases and/or cancer indications in a mammal suffering from any one or more of the above-identified cancer diseases or cancer indications which comprises administering to the mammal in need thereof a therapeutically effective pain or symptom-reducing amount of a pharmaceutical composition comprising effective amounts of a substance exhibiting Tubercin and/or SSM activity and/or Z-100 activity or a finctional derivative thereof and a pharmaceutically acceptable excipient, either alone or in combination with one or more anti-inflammatory compounds or immunomodulatory agents, wherein said substance exhibiting Tubercin and/or SSM activity and/or Z-100 activity or a functional derivative thereof is sufficient to inhibit the cancer or tumor development, metastasis, protein degradation, and/or cell proliferation in cancer cells.

The effective dose of the agent of the invention, and the appropriate treatment regime, can vary with the indication and patient condition, and the nature of the molecule itself, e.g., its in vivo half life and level of activity. These parameters are readily addressed by one of ordinary skill in the art and can be determined by routine experimentation.

The preferred doses for administration can be anywhere in a range between about 1 picogram/ml and about 500 ug/ml of biologic fluid of treated patient. The therapeutically effective amount of the substance exhibiting Tubercin and/or S SM activity and/or Z-100 or a functional derivative thereof that has similar antiviral activities as substance exhibiting Tubercin and/or SSM activity and/or Z-100 or a functional derivative thereof can be also measured in molar concentrations and can range between about 1 nM to about 2 mM.

Cancer Diseases and/or Symptoms Treatable by the Methods of the Present Invention

In one aspect, the cancer diseases and cancer disorders that may be treated using the Tubercin and/or SSM activity and/or Z-100 or a functional derivatives thereof of the present invention include for example, an not by way of limitation, AIDS-Associated Cancers, Bladder Cancer, Bone Cancer, Brain & Spinal Cord Cancers, Metastatic Brain Tumors, Pediatric Brain Tumors, Breast Cancer, Male Breast Cancer, Cervical Cancer, Colorecfal Cancer, Endometrial & Other Uterine Cancers, Esophageal Cancer, Gallbladder & Bile Duct Cancers, Gastric (Stomach) Cancer, Gestational Trophoblastic Disease, Head & Neck Cancers, Kidney Cancer, Leukemia, Liver Cancer, Liver Metastases, Lung Cancer, Lymphomas, Melanoma, Multiple Myeloma, Myelodysplastic Syndrome, Neuroblastoma, Ovarian Cancer, Pancreatic Cancer, Pediatric Cancers, Pituitary Tumors, Prostate Cancer, Rare Hematologic Disorders, Rare Solid Tumors, Retinoblastoma, Skin Cancer, Soft-Tissue Sarcoma, Testicular Cancer, Thyroid Cancer, Uterine Cancers, Wilms' Tumor, Bronchus Cancer, Colon and Rectum Cancer, Urinary Cancer, Non-Hodgkin Lymphoma, Melanomas of the Skin, Kidney and Renal Cancer, Pelvis Cancer, Pancreatic Cancer, Oral Cavity Cancer and Pharynx Cancer, Ovary Cancer, Stomach Esophagus Cancer, Intrahepatic Bile Duct Cancer, Cervix Cancer, Larynx Cancer, Acute Myeloid Leukemia, Chronic Lymphocytic Leukemia, Soft Tissue Cancer including Heart, GIST (Gastro-Intestinal Stromal Tumors), Small Intestine Cancer, Chronic Myeloid Leukemia, Acute Lymphocytic Leukemia Anus, Anal, Canal and Anorectum Cancers, Vulva Cancer, Gallbladder Cancer, Pleura Cancer, Malignant Mesothelioma, Cancer of the Bones and Joints, Hypopharynx Cancer, Eye and Orbit Cancer, Nose and Nasal Cavity Cancer, Middle Ear Cancer, Nasopharynx Cancer, Ureter Cancer, Peritoneum Cancer, Omentum and Mesentery Cancer, and Gastrointestinal Carcinoid Tumors or any combination thereof.

In yet another aspect, the cancer diseases and cancer disorders that may be treated using the Tubercin and/or SSM activity and/or Z-100 or a functional derivatives thereof of the present invention include for example, an not by way of limitation, colorectal cancer, gastric cancer, ovarian cancer, osteosarcoma, hepatocellular carcinoma, Burkitt's lymphoma, primary effuision lymphomas, angioimmunoblastic lymphadenopathy, acquired immune deficiency syndrome (AIDS)-related lymphoma, T-cell lymphomas, oral hairy leukoplakia, lymphoproliferative disease, lymphoepithelial carcinoma, body-cavity-based lymphoma or B-cell lymphomas, non-keratinising carcinoma, squamous cell nasopharyngeal carcinoma, kidney transplant-associated epithelial tumors, angiosarcoma, Kaposi's sarcoma, angiolymphoid hyperplasia, prostatic neoplasm, retinoblastoma, Li-Fraumeni syndrome, Gardner's syndrome, Werner's syndrome, nervoid basal cell carcinoma syndrome, neurofibromatosis type 1, cervical dysplasia, neuro-blastoma, primary macroglobulinemia, insulinoma, mycosis fungoides, osteogenic sarcoma, premalignant skin lesions (topical), rhabdomyosarcoma, osteogenic, polycythemia vera, essential thrombocytosis or any combination thereof.

Compositions for Use in the Methods of the Present Invention

Tubercin

Tubercin-3 and Tubercin-5

The Tubercin compositions that may be used in the methods of the present invention comprises those tubercin carbohydrate complexes designated Tubercin-3 and Tubercin-5. Tubercin-3 may be prepared as described in Chung, T. H., J. Korean Med. Ass., 17, 427-431(1974); Chung, T. H. et al., Yonsei Med. J., 17, 131-135(1976). Tubercin-5 may be extracted from M. tuberculosis as described in U.S. Pat. No. 6,274,356, the full contents of which are incorporated herein by reference. Tubercin-5 is a mixture of polysaccharides having straight-chain and side-chain glycosidic bonds formed between such essential monosaccharides as mannose, arabinose, glucose and galactose. The molecular weight of said polysacchrides lies below 7,000, preferably in the range of 2,500 to 3,500 dalton.

For example, the Tubercin-5 carbohydrate complex consists of an extract of Mycobacterium tuberculosis consisting essentially of polysaccharides comprising mannose, arabinose, glucose and galactose as constituents thereof, wherein the weight average molecular weight of each polysaccharide is 7,000 or less and the partial acid hydrolysis product of the polysacharides comprises: 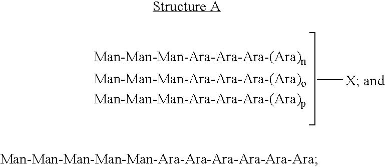
wherein n, o and p are individually an integer; and x is a chain of glucose and galactose residues.

In another embodiment, the partial acid hydrolysis product of the extract of Tubercin-5 depicted in Structure A can further comprise 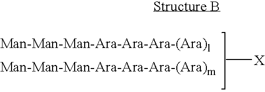
wherein 1 and m are individually an integer; and x is a chain of glucose and galactose residues.

In yet another embodiment, the partial acid hydrolysis product of the extract of Tubercin-5 depicted in Structure B can comprise

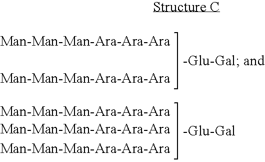

In yet another embodiment, the partial acid hydrolysis product of the extract of Tubercin-5 depicted in Structure A can further comprise partial acid hydrolysis products of any of the above, including, but not limited to:
Man-Man;
Man-Man-Man;
Man-Man-Man-Ara;
Man-Man-Man-Ara-Ara:,
Man-Man-Man-Man-Ara-Ara-Ara-Ara;
Man-Man-Man-Ara-Ara-Ara-Ara-Ara-Ara; and
Man-Man-Man-Man-Ara-Ara-Ara-Ara-Ara-Ara.
   Specific Substance of Maruyama (SSM)

SSM refers to a vaccine derived from a strain of tuberculosis, which gets its name, Specific Substance of Maruyama, from the late Professor Chisato Maruyama of Tokyo's Nippon Medical School. SSM (sometimes referred to herein as SSMA) comprises arabinomannan as a polysaccharide and has fatty acids bonded to said arabinomannan through an ester linkage, the fatty acid content in said lipopolysaccharide being from 3 to 28%. The lipopolysaccharide is obtained by hot water extraction and purification of the cell body of human tubercle bacillus, Mycobacterium tuberculosis strain Aoyama B or Mycobacterium tuberculosis strain H₃₇ Rᵥ SSM is thus a lipoarabinomannan and comprises a lipopolysaccharide structure having definite chemical composition, which may be isolated and purified as described in either U.S. Pat. No. 4,394,502, the full contents of which are incorporated herein by reference, or U.S. Pat. No. 4,329,452, the full contents of which are incorporated herein by reference. In some embodiments, the SSM lipopolysaccharide may be prepared by bonding a fatty acid to arabinomannan through an ester linkage, said arabinomannan being obtained by alkali extraction and purification of the cell body of human tubercle bacillus, Mycobacterium tuberculosis strain Aoyama B or Mycobacterium tuberculosis strain H₃₇ Rᵥ, the fatty acid content in said lipopolysaccharide being 3 to 28%. In other embodiments, the SSM lipopolysaccharide may be prepared by bonding a fatty acid to lipoarabinomannan through an ester linkage, said lipoarabinomannan being obtained by hot water extraction and purification of the cell body of human tubercle bacillus, Mycobacterium tuberculosis strain Aoyama B or Mycobacterium tuberculosis strain H₃₇ Rᵥ, the fatty acid content in said lipopolysaccharide being 3 to 28%. In either case, the fatty acids are palmitic acid, myristic acid, stearic acid, tuberculostearic acid, heptadecanoic acid, oleic acid and linoleic acid and said lipopolysaccharide has a monosaccharide composition of 30 to 74% of arabinose, 20 to 50% of mannose, 0 to 10% of glucose and 0 to 13% of galactose.

In a study to examine the effect of SSM on clearance of HBe antigen, Satomura K et al. demonstrated that SSM stimulates the production of IFN-gamma in human peripheral blood cells and also suggest that treatment of HBe antigen-positive chronic hepatitis B patients with SSM leads to the clearance of HBe antigen and normalization of serum aspartate aminotransferase levels through inhibition of IL-10 and stimulation of IFN-gamrnma. (Effects of SSM (specific substance maruyama) on HBe antigen-positive chronic hepatitis B -clinical efficacy and modulation of cytokines J Nippon Med Sch. 2000 Aug; 67(4):261-6). Z-100 is a more powerful version of SSM. Z-100 is the same agent as SSM, but used in different concentrations. Z-100 is an arabinomannan extracted from Mycobacterium tuberculosis that has various immunomodulatory activities, such as the induction of interleukin 12, interferon gamma (IFN-) and -chemokines (Inhibition of human immunodeficiency virus type 1 replication by Z-100, an immunomodulator extracted from human-type tubercle bacilli, in macrophages Yutaka Emoril et al. J Gen Virol 85 (2004), 2603-2613). Yutaka et al. investigated the effects of Z-100 on human immunodeficiency virus type 1 (HIV-1) replication in human monocyte-derived macrophages (MDMs). In MDMs, Z-100 markedly suppressed the replication of not only macrophage-tropic (M-tropic) HIV-1 strain (HIV-1JR-CSF), but also HIV-1 pseudotypes that possessed amphotropic Moloney murine leukemia virus or vesicular stomatitis virus G envelopes. Yutaka et al. also demonstrated that Z-100 was found to inhibit HIV-1 expression, even when added 24 h after infection. In addition, Yutaka et al. also demonstrated that Z-100 substantially inhibited the expression of the pNL43lucenv vector (in which the env gene is defective and the nef gene is replaced with the firefly luciferase gene) when this vector was transfected directly into MDMs. Together, these findings suggest that Z-100 inhibits virus replication, mainly at the level of HIV-1 transcription. However, Yutaka et al. also demonstrated that Z-100 also downregulated expression of the cell surface receptors CD4 and CCR5 in MDMs, suggesting some inhibitory effect on HIV-1 entry. Further experiments by Yutaka et al. demonstrated that Z-100 revealed that Z-100 induced IFN-production in these cells, resulting in induction of the 16-kDa CCAAT/enhancer binding protein (C/EBP) transcription factor that represses HIV-1 long terminal repeat transcription. These effects were alleviated by SB 203580, a specific inhibitor of p38 mitogen-activated protein kinases (MAPK), indicating that the p38 MAPK signalling pathway was involved in Z-100-induced repression of HIV-1 replication in MDMs. Taken together, these findings of Yutaka et al. suggest that Z-100 might be a useful immunomodulator for control of HIV-1 infection.

Moreover, the effect of Z-100, a lipid-arabinomannan extracted from Mycobacterium tuberculosis strain Aoyama B, was investigated on the resistance of thermally injured mice (TI-mice) to herpes simplex virus type 1 (HSV) infections Z-100 (Kobayashi M et al. Lipid-arabinomannan extracted from Mycobacterium tuberculosis, improves the resistance of thermally injured mice to herpes virus infections (Kobayashi M et al. Immunol Lett. 1994 Jun;40(3):199-205). Kobayashi M et al. demonstrated that the susceptibility of TI mice to infection was about 100 times greater than it was in normal mice (N mice). However, the increased susceptibility of TI mice to infection was effectively counteracted to the levels observed in N mice when treated with Z-100 (10 mg/kg i.p.; 1,3 and 5 days after thermal injury). Kobayashi M et al. demonstrated that adoptive transfer of burn-associated CD8+CD11b+TCR gamma/delta+suppressor T (BAST) cells, prepared from TI mice, increased the susceptibility of N mice to infection by HSV, while the susceptibility of N mice, inoculated with the CD8+T-cell fraction prepared from Z-100-treated TI mice (ZTC), to infection was not changed. In addition, Kobayashi M et al. demonstrated that the suppressor cell activity of BAST cells was not demonstrated when they were assayed in vitro in the presence of anti-IL-4 monoclonal antibody (mAb). BAST cells released IL-4 into their culture fluids without stimulation. The suppressor cell activity of ZTC and IL-4 production by ZTC were minimal. Taken together, the results of Kobayashi M et al. demonstrated that Z-100 may improve the resistance of TI mice to HSV infection through the regulation of BAST cells and/or the release of IL-4 from these cells.

In addition to the Tubercin and SSM (Z-100) compounds and/or substances disclosed supra, in yet another embodiment, the SSM and/or Tubercin and/or Z-100 composition for use in all of the aforementioned methods of the present invention may be a SSM and/or Tubercin and/or Z-100 functional derivative composition comprising, inter alia, a polysaccharide produced by a hot aqueous solvent extraction of tubercle bacillus, wherein the polysaccharide is comprised of arabinose, mannose and glucose residues. The SSM and/or Tubercin and/or Z-100 functional derivative composition for use in the methods of the present invention may be further described in that the polysaccharide has a molecular weight of about 5.times.10.sup.2 -5.times.10.sup.4, as determined by gel filtration. In one embodiment, the polysaccharide of the SSM/Tubercin/Z-100 functional derivative composition of the present invention is comprised of 10-72 wt. % mannose, 3-30 wt. % of arabinose and 5-30% wt. % of glucose. In another embodiment, the polysaccharide of the SSM/Tubercin/Z-100 functional derivative composition of the present invention is comprised of 40-50 wt. % mannose, 15-25 wt. % of arabinose and 5-15% wt. % of glucose. The SSM/Tubercin/Z-100 functional derivative composition may be prepared and isolated as more fully described in U.S. Pat. No. 6,015,796. It is intended herein that the ranges recited also include all those specific percentage amounts between the recited range. For example, the range of about 10 to 72% also encompasses 11 to 71%, 12 to 708%, etc, without actually reciting each specific range therewith.

Additional Combination Therapies for Treating Cancer Diseases or Cancer Disorders Using the Methods of the Invention

In each of the aforementioned aspects and embodiments of the invention, combination therapies other than those enumerated above are also specifically contemplated herein.

In particular, the compositions of the present invention may be administered with one or more of the currently available chemotherapeutic agents according to class, and including diseases for which the agents are indicated, is provided below as Table 1.

TABLE 1
Neoplastic Diseases for which Exemplary Chemotherapeutic agents are Indicated

| Class | Type of Agent | Name | Disease |
|---|---|---|---|
| Alkylating | Nitrogen Mustards | Mechlorethamine | Hodgkin's disease, non- |
| Agents | (HN₂) | | Hodgkin's lymphomas Acute |
| | | | and chronic |
| | Cyclophosphamide | | lymphocytic leukemias, |
| | Ifosfamide | | Hodgkin's disease, |
| | | | non-Hodgkin's lymphomas, |
| | | | multiple myeloma, neuro- |
| | | | blastoma, breast, ovary, |
| | | | lung, Wilms' tumor, cervix, |
| | | | testis, soft-tissue sarcomas |
| | Melphalan | | Multiple myeloma, breast, ovary |
| | Chlorambucil | | Chronic lymphocytic leukemia, |
| | | | primary macroglobulinemia, |
| | | | Hodgkin's disease, |
| | | | non-Hodgkin's lymphomas |
| | Estramustine | | Prostate |
| | Ethylenimines and | Hexamethylmelamine | Ovary |
| | Methylmelamines | Thiotepa | Bladder, breast, ovary |
| | Alkyl Sulfonates | Busulfan | Chronic granulocytic leukemia |
| | Nitro- | Carmustine | Hodgkin's disease, |
| | | | non-Soureas Hodgkin's |
| | | | lymphomas, primary brain tumors, |
| | | | multiple myeloma, malignant |
| | | | melanoma |
| | Lomustine | | Hodgkin's disease, |
| | | | non-Hodgkin's lymphomas, |
| | | | primary brain tumors, small-cell |
| | | | lung |
| | Semustine | | Primary brain tumors, stomach, |
| | | | colon |
| | Streptozocin | | Malignant pancreatic insulinoma, |
| | | | malignant carcinoid |
| | Triazenes | Dacarbazine | Malignant melanoma, |
| | Procarbazine | | Hodgkin's disease, |
| | Aziridine | | soft-tissue sarcomas |
| metabolites | Anti-Folic Acid | Methotrexate | Acute lymphocytic leukemia, |
| | Analogs | Trimetrexate | chorio-carcinoma, mycosis |
| | | | fungoides, breast, head and |
| | | | neck, lung, osteogenic sarcoma |
| | Pyrimidine Analogs | Fluorouracil | Breast, colon, stomach, |
| | | Floxuridine | pancreas, ovary, head and neck, |
| | | | urinary bladder, premalignant |
| | | | skin lesions (topical) |
| | | Cytarabine | Acute granulocytic and acute |
| | | Azacitidine | lymphocytic leukemias |
| | Purine Analogs and | Mercaptopurine | Acute lymphocytic, acute |
| | Related Inhibitors | | granulocytic, and chronic |
| | | | granulocytic leukemias |
| | | Thioguanine | Acute granulocytic, acute |
| | | | lymphocytic, and chronic |
| | | | granulocytic leukemias |
| | | Pentostatin | Hairy cell leukemia, mycosis |
| | | | fungoides, chronic lymphocytic |
| | | | leukemia |
| | | Fludarabine | Chronic lymphocytic leukemia, |
| | | | Hodgkin's and non-Hodgkin's |
| | | | lymphomas, mycosis fungoides |
| Natural | Vinca Alkaloids | (VLB) | Vinblastine Hodgkin's |
| Products | | | disease, non Hodgkin's |
| | | | lymphomas, breast, testis |
| | | Vincristine | Acute lymphocytic |
| | | Vindesine | Vinca-resistant acute |
| | | | lymphocytic leukemia, |
| | | | chronic myelocytic leukemia, |
| | | | melanoma, lymphomas, breast |
| | Epipodophyllotoxins | Etoposide Teniposide | Testis, small-cell lung and |
| | | | other lung, breast Hodgkin's |
| | | | disease, non-Hodgkin's |
| | | | lymphomas, acute granulocytic |
| | | | leukemia, Kaposi's sarcoma |
| | Antibiotics | Dactinomycin | Choriocarcinoma, Wilms' |
| | | | tumor, rhabdomyosarcoma, |
| | | | testis Kaposi's sarcoma |
| | | Daunorubicin | Acute granulocytic and acute |
| | | | lymphocytic leukemias |
| | Doxorubin | | Soft-tissue, osteogenic, and |
| | 4′-Deoxydoxorubicin | | other sarcomas; Hodgkin's |
| | | | disease, non-Hodgkin's |
| | | | ymphomas, acute leukemias, |
| | | | breast, genitourinary, thyroid, |
| | | | lung, stomach, neuroblastoma |
| | Bleomycin | | Testis, head and neck, skin, |
| | | | esophagus, lung, and |
| | | | genitourinary tract; Hodgkin's |
| | | | disease, non-Hodgkin's lymphomas |
| | Plicamycin | | Testis, malignant, hypercalcemia |
| | Mitomycin | | Stomach, cervix, colon, breast, |
| | | | pancreas, bladder, head and neck |
| Enzymes | L-Asparaginase | | Acute lymphocytic leukemia |
| | Taxanes | Docetaxel | Breast, ovarian |
| | Taxoids | Paclitaxel | |
| Biological | Interferon Alfa | | Hairy cell leukemia, Kaposi's |
| Response | | | sarcoma, melanoma, carcinoid, |
| Modifiers | | | renal cell, ovary, bladder, |
| | | | non-Hodgkin's lymphomas, |
| | | | mycosis fungoides, multiple |
| | | | myeloma, chronic granutocytic |
| | | | leukemia |
| | Tumor Necrosis | | Investigational |
| | Factor | | |
| | Tumor-Infiltrating | | Investigational |
| | Lymphocytes | | |
| Miscellaneous | Platinum | Cisplatin | Testis, ovary, bladder, head |
| Agents | Coordination | Carboplatin | and neck, lung, thyroid, cervix, |
| | Complexes | | endometrium, neuroblastoma, |
| | | | osteogenic sarcoma |
| | Anthracenedione | Mitoxantrone | Acute granulocytic leukemia, |
| | | | breast |
| | Substituted Urea | Hydroxyurea | Chronic granulocytic leukemia, |
| | | | polycythemia vera, essential |
| | | | thrombocytosis, malignant |
| | | | melanoma |
| | Methyl Hydrazine | Porcarbazine | Hodgkin's disease |
| | Derivative | | |
| | Adrenocortical | Mitotane | Adrenal cortex |
| | Suppressant | | |
| | | Amino- | Breast |
| | | glutethimide | |
| Hormones and | Adrenocorti- | Prednisone | Acute and chronic lymphocytic |
| Antagonists | costeroids | | leukemias, non-Hodgkin's |
| | | | lymphomas, Hodgkin's disease, |
| | | | breast |
| | Progestins | Hydroxyprogesterone | Endometrium, breast |
| | | caproate | |
| | | Medroxyprogesterone | |
| | | acetate | |
| | | Megestrol Acetate | |
| | Estrogens | Diethylstilbestrol | Breast, prostate |
| | | Ethinyl Estradiol | |
| | Antiestrogen | Tamoxifen | Breast |
| | Androgens | Testosterone | Breast |
| | | propionate | |
| | | Fluoxy-Mesterone | |
| | Anti-androgen | Flutamide | Prostate |
| | Gonadotropin- | Leuprolide Goserelin | Prostate, Estrogen-receptor- |
| | releasing hormone | | positive breast |
| | analog | | |

In particular, the compositions of the present invention may be administered with one or more macrolide or non-macrolide antibiotics, anti-bacterial agents, anti-fungicides, anti-viral agents, and anti-parasitic agents, anti-inflammatory or immunomodulatory drugs or agents.

Examples of macrolide antibiotics that may be used in combination with the composition of the present invention include, inter alia, the following synthetic, semi-synthetic or naturally occurring microlidic antibiotic compounds: methymycin, neomethymycin, YC-17,litorin, erythromycin A to F, oleandomycin, roxithromycin, dirithromycin, flurithromycin, clarithromycin, davercin, azithromycin, josamycin, kitasamycin, spiramycin, midecamycin, rokitamycin, miokamycin, lankacidin, and the derivatives of these compounds. Thus, erythromycin and compounds derived from erythromycin belong to the general class of antibiotics known as “macrolides.” Examples of preferred erythromycin and erythromycin-like compounds include: erythromycin, clarithromycin, azithromycin, and troleandomycin.

Additional antibiotics, other than the macrolidic antibiotics described above, which are suitable for use in the methods of the present invention include, for example, any molecule that tends to prevent, inhibit or destroy life and as such, and as used herein, includes anti-bacterial agents, anti-fuingicides, anti-viral agents, and anti-parasitic agents. These agents may be isolated from an organism that produces the agent or procured from a commercial source (e.g., pharmaceutical company, such as Eli Lilly, Indianapolis, Ind.; Sigma, St. Louis, Mo.).

For example, the anti-TB antibiotic isoniazid (isonicotinic acid hydrazide) is frequently effective, but isoniazid often causes severe, sometimes fatal, hepatitis. The risk of hepatitis increases with the patient's age. Additionally, isoniazid causes peripheral neuropathy in some recipients in a dose-related fashion. Rifampin, another antibiotic used to treat TB, must be used in conjunction with another drug such as isoniazid. This requirement for combination therapy with rifampin applies to the initial treatment as well as the retreatment of pulmonary TB.

Usually, isoniazid, rifampin, ethambutol and ethionamide are given orally. Streptomycin is typically given intramuscularly. Amikacin is given intramuscularly or intravenously. Clofazimine, which is also used to treat leprosy, is given orally.

Amikacin is a semisynthetic aminoglycoside antibiotic derived from Kanamycin A. For its preparation see U.S. Pat. No. 3,781,268. For a review see Kerridge, Pharmacological and Biochemical Properties of Drug Substances 1:125-153, M. E. Goldberg, ed. (1977). Amikacin is usually administered intramuscularly or intravenously. For additional information including clinical pharmacology, indications, side effects and dosages, see the Physicians Desk Reference, 42 ed. (1988) at pages 744-746 (hereinafter, PDR).

Clofazimine is an antibacterial agent also known as LAMPRENE.RTM. For its preparation, see Barry, et at., Nature 179:1013 (1957). For a review see Karat, et al., Brit. Med. J. 3:175 (1971). Clofazimine is generally given orally. For additional information including clinical pharmacology, precautions and dosages, see the PDR at page 982. Ethionamide is an antibacterial agent also known as AMIDAZINE.RTM. and TRECATOR.RTM. See British Patent No. 800,250. This drug is typically given orally. For further information including precautions and dosages, see the PDR at page 2310.

Ciprofloxacin is a broad spectrum synthetic antibacterial agent for oral usage. It is also known as CIPRO.RTM. It is typically given in total daily dosages of 500 to 1,000 milligrams which is usually given in 2 equal doses in 24 hours. For further information see the PDR (1989) at pages 1441-1443. other member of this fluoroquinolone class of antibiotics include ofloxacin, levofloxacin, troveofloxacin, pefloxacin, gatifloxacin, and moxifloxacin.

Other examples of anti-bacterial antibiotic agents include, but are not limited to, penicillins, cephalosporins, carbacephems, cephamycins, carbapenems, monobactams, aminoglycosides, glycopeptides, quinolones, tetracyclines, macrolides, oxazalidinones, and fluoroquinolones. Examples of antibiotic agents include, but are not limited to, Penicillin G (CAS Registry No.: 61 -33-6); Methicillin (CAS Registry No.: 61-32-5); Nafcillin (CAS Registry No.: 147-52-4); Oxacillin (CAS Registry No.: 66-79-5); Cloxacillin (CAS Registry No.: 61-72-3); Dicloxacillin (CAS Registry No.: 3116-76-5); Ampicillin (CAS Registry No.: 69-53-4); Amoxicillin (CAS Registry No.: 26787-78-0); Ticarcillin (CAS Registry No.: 34787-01-4); Carbenicillin (CAS Registry No.: 4697-36-3); Mezlocillin (CAS Registry No.: 51481-65-3); Azlocillin (CAS Registry No.: 37091-66-0); Piperacillin (CAS Registry No.: 61477-96-1); Imipenem (CAS Registry No.: 74431-23-5); Aztreonam (CAS Registry No.: 78110-38-0); Cephalothin (CAS Registry No.: 153-61-7); Cefazolin (CAS Registry No.: 25953-19-9); Cefaclor (CAS Registry No.: 70356-03-5); Cefamandole formate sodium (CAS Registry No.: 42540-40-9); Cefoxitin (CAS Registry No.: 35607-66-0); Cefuroxime (CAS Registry No.: 55268-75-2); Cefonicid (CAS Registry No.: 61270-58-4); Cefmetazole (CAS Registry No.: 56796-20-4); Cefotetan (CAS Registry No.: 69712-56-7); Cefprozil (CAS Registry No.: 92665-29-7); Loracarbef (CAS Registry No.: 121961-22-6); Cefetamet (CAS Registry No.: 65052-63-3); Cefoperazone (CAS Registry-No.: 62893-19-0); Cefotaxime (CAS Registry No.: 63527-52-6); Ceftizoxime (CAS Registry No.: 68401-81-0); Ceftriaxone (CAS Registry No.: 73384-59-5); Ceftazidime (CAS Registry No.: 72558-82-8); Cefepime (CAS Registry No.: 88040-23-7); Cefixime (CAS Registry No.: 79350-37-1); Cefpodoxime (CAS Registry No.: 80210-62-4); Cefsulodin (CAS Registry No.: 62587-73-9); Fleroxacin (CAS Registry No.: 79660-72-3); Nalidixic acid (CAS Registry No.: 389-08-2); Norfloxacin (CAS Registry No.: 70458-96-7); Ciprofloxacin (CAS Registry No.: 85721-33-1); Ofloxacin (CAS Registry No.: 82419-36-1); Enoxacin (CAS Registry No.: 74011-58-8); Lomefloxacin (CAS Registry No.: 98079-51-7);° Cinoxacin (CAS Registry No.: 28657-80-9); Doxycycline (CAS Registry No.: 564-25-0); Minocycline (CAS Registry No.: 10118-90-8); Tetracycline (CAS Registry No.: 60-54-8); Amikacin (CAS Registry No.: 37517-28-5); Gentamicin (CAS Registry No.: 1403-66-3); Kanamycin (CAS Registry No.: 8063-07-8); Netilmicin (CAS Registry No.: 56391-56-1); Tobramycin (CAS Registry No.: 32986-56-4); Streptomycin (CAS Registry No.: 57-92-1); Azithromycin (CAS Registry No.: 83905-01-5); Clarithromycin (CAS Registry No.: 81103-11-9); Erythromycin (CAS Registry No.: 114-07-8); Erythromycin estolate (CAS Registry No.: 3521-62-8); Erythromycin ethyl succinate (CAS Registry No.: 41342-53-4); Erythromycin glucoheptonate (CAS Registry No.: 23067-13-2); Erythromycin lactobionate (CAS Registry No.: 3847-29-8); Erythromycin stearate (CAS Registry No.: 643-22-1); Vancomycin (CAS Registry No.: 1404-90-6); Teicoplanin (CAS Registry No.: 61036-64-4); Chloramphenicol (CAS Registry No.: 56-75-7); Clindamycin (CAS Registry No.: 18323-44-9); Trimethoprim (CAS Registry No.: 738-70-5); Sulfamethoxazole (CAS Registry No.: 723-46-6); Nitrofurantoin (CAS Registry No.: 67-20-9); Rifampin (CAS Registry No.: 13292-46-1); Mupirocin (CAS Registry No.: 12650-69-0); Metronidazole (CAS Registry No.: 443-48-1); Cephalexin (CAS Registry No.: 15686-71-2); Roxithromycin (CAS Registry No.: 80214-83-1); Co-amoxiclavuanate; combinations of Piperacillin and Tazobactam; and their various salts, acids, bases, and other derivatives.

Anti-fungal agents include, but are not limited to, caspofingin, terbinafine hydrochloride, nystatin, amphotericin B, griseofulvin, ketoconazole, miconazole nitrate, flucytosine, fluconazole, itraconazole, clotrimazole, benzoic acid, salicylic acid, and selenium sulfide.

Anti-viral agents include, but are not limited to, valgancyclovir, amantadine hydrochloride, rimantadin, acyclovir, famciclovir, foscarnet, ganciclovir sodium, idoxuridine, ribavirin, sorivudine, trifluridine, valacyclovir, vidarabin, didanosine, stavudine, zalcitabine, zidovudine, interferon alpha, and edoxudine.

Anti-parasitic agents include, but are not limited to, pirethrins/piperonyl butoxide, permethrin, iodoquinol, metronidazole, diethylcarbamazine citrate, piperazine, pyrantel pamoate, mebendazole, thiabendazole, praziquantel, albendazole, proguanil, quinidine gluconate injection, quinine sulfate, chloroquine phosphate, mefloquine hydrochloride, primaquine phosphate, atovaquone, co-trimoxazole (sulfamethoxazole/trimethoprim), and pentamidine isethionate.

In another aspect, in the method of the present invention, one may, for example, supplement the composition by administration of a therapeutically effective amount of one or more an anti-inflammatory or immunomodulatory drugs or agents. By “immunomodulatory drugs or agents”, it is meant, e.g., agents which act on the immune system, directly or indirectly, e.g., by stimulating or suppressing a cellular activity of a cell in the immune system, e.g., T-cells, B-cells, macrophages, or antigen presenting cells (APC), or by acting upon components outside the immune system which, in turn, stimulate, suppress, or modulate the immune system, e.g., hormones, receptor agonists or antagonists, and neurotransmitters; immunomodulators can be, e.g., immunosuppressants or immunostimulants. By “anti-inflammatory drugs”, it is meant, e.g., agents which treat inflammatory responses, i.e., a tissue reaction to injury, e.g., agents which treat the immune, vascular, or lymphatic systems.

Anti-inflammatory or immunomodulatory drugs or agents suitable for use in this invention include, but are not limited to, interferon derivatives, e.g., betaseron, .beta.-interferon; prostane derivatives, e.g., compounds disclosed in PCT/DE93/0013, e.g., iloprost, cicaprost; glucocorticoid, e.g., cortisol, prednisolone, methylprednisolone, dexamethasone; immunsuppressives, e.g., cyclosporine A, FK-506, methoxsalene, thalidomide, sulfasalazine, azathioprine, methotrexate; lipoxygenase inhibitors, e.g., zileutone, MK-886, WY-50295, SC-45662, SC-41661A, BI-L-357; leukotriene antagonists, e.g., compounds disclosed in DE 40091171 German patent application P 42 42 390.2; WO 9201675; SC-41930; SC-50605; SC-51146; LY 255283 (D. K. Herron et al., FASEB J. 2: Abstr. 4729, 1988); LY 223982 (D. M. Gapinski et al. J. Med. Chem. 33: 2798-2813, 1990); U-75302 and analogs, e.g., described by J. Morris et al., Tetrahedron Lett. 29: 143-146, 1988, C. E. Burgos et al., Tetrahedron Lett. 30: 5081-5084, 1989; B. M. Taylor et al., Prostaglandins 42: 211-224, 1991; compounds disclosed in U.S. Pat. No. 5,019,573; ONO-LB-457 and analogs, e.g., described by K. Kishikawa et al., Adv. Prostagl. Thombox. Leukotriene Res. 21: 407-410, 1990; M. Konno et al., Adv. Prostagl. Thrombox. Leukotriene Res. 21: 411-414, 1990; WF-11605 and analogs, e.g., disclosed in U.S. Pat. No. 4,963,583; compounds disclosed in WO 9118601, WO 9118879; WO 9118880, WO 9118883, antiinflammatory substances, e.g., NPC 16570, NPC 17923 described by L. Noronha-Blab. et al., Gastroenterology 102 (Suppl.): A 672, 1992; NPC 15669 and analogs described by R. M. Burch et al., Proc. Nat. Acad. Sci. USA 88: 355-359, 1991; S. Pou et al., Biochem. Pharmacol. 45: 2123-2127, 1993; peptide derivatives, e.g., ACTH and analogs; soluble TNF-receptors; TNF-antibodies; soluble receptors of interleukines, other cytokines, T-cell-proteins; antibodies against receptors of interleukins, other cytokines, and T-cell-proteins.

In yet another aspect of the present invention, for each of the above-recited methods, the one or more substances exhibiting Tubercin and/or SSM activity or a functional derivative thereof may themselves be administered as an adjuvant, wherein the therapeutically effective amount of the one or more substances exhibiting Tubercin and/or SSM activity or a functional derivative thereof acts as an immunostimulant or immunomodulator to be used either alone or in conjunction with one or more of the pharmaceutical drugs listed infra.

In yet another aspect of the present invention, for each of the above-recited methods of the present invention, the therapeutically effective amount of the one or more substances exhibiting Tubercin and/or SSM activity and/or Z-100 or a functional derivative thereof may themselves be administered as an adjuvant in vaccine preparations to improve vaccine responses to all known bacterial, viral or parasitic antigen preparations, wherein the therapeutically effective amount of the one or more substances exhibiting Tubercin and/or SSM activity and/or Z-100 or a functional derivative thereof acts as an immunostimulant or immunomodulator to be used either alone or in conjunction with one or more of the pharmaceutical drugs. Representative examples of pharmaceutical drugs that may be used in accordance with the of the present invention include the following, as well as each specific indication specifically listed for use with each drug as if set forth herein in their entirety and as described in the Physician's Desk Reference (PDR)(the entire contents of which are incorporated herein by reference) listed in the Physician's Desk Reference.

The therapeutic agents of the instant invention may be used for the treatment of animal subjects or patients, and more preferably, mammals, including humans, as well as mammals such as non-human primates, dogs, cats, horses, cows, pigs, guinea pigs, and rodents.

Fusion Proteins

In each of the aforementioned aspects and embodiments of the invention, fusion polypeptides are also specifically contemplated herein.

In one embodiment, fusion polypeptides of the invention are produced by recombinant DNA techniques. Alternative to recombinant expression, a fusion polypeptide of the invention can be synthesized chemically using standard peptide synthesis techniques. The present invention also provides compositions that comprise a fusion polypeptide of the invention and a pharmaceutically acceptable carrier, excipient or diluent.

In each of the above-recited methods, the substance exhibiting Tubercin and/or SSM activity and/or Z-100 or a functional derivative thereof may be part of a fusion polypeptide, wherein said fusion polypeptide or conjugate fusion polypeptide comprises a substance exhibiting Tubercin and/or SSM activity and/or Z-100 or a functional derivative thereof and an amino acid sequence heterologous to said substance exhibiting Tubercin and/or SSM activity and/or Z-100 or a functional derivative thereof.

Among the particular fusion polypeptides or conjugate fusion polypeptides of the invention are, for example, fusion polypeptides or conjugate fusion polypeptides that comprise the substance exhibiting Tubercin and/or SSM activity and/or Z-100 or a functional derivative thereof.

The fusion polypeptides or conjugate fusion polypeptides of the invention can be such that the heterologous amino acid sequence comprises a human immunoglobulin constant region, such as a human IgGI constant region, including a modified human IgGI constant region wherein the IgGI constant region does not bind Fc receptor and/or does not initiate antibody-dependent cellular cytotoxicity (ADCC) reactions.

In particular, in one embodiment the fusion protein or conjugate fusion polypeptide comprises a heterologous sequence that is a sequence derived from a member of the immunoglobulin protein family, for example, comprise an immunoglobulin constant region, e.g., a human immunoglobulin constant region such as a human IgG1 constant region. The fusion protein or conjugate fusion polypeptide can, for example, comprise a portion of a substance exhibiting Tubercin and/or SSM activity or a functional derivative thereof fused or conjugated with the amino-terminus or the carboxyl-terminus of an immunoglobulin constant region, as disclosed, e.g., in U.S. Pat. Nos. 5,714,147, 5,116,964, 5,514,582, and 5,455,165. In those embodiments in which all or part of a substance exhibiting Tubercin and/or SSM activity or a functional derivative thereof of the invention is fused with sequences derived from a member of the immnunoglobulin protein family, the FcR region of the immunoglobulin may be either wild-type or mutated. In certain embodiments, it is desirable to utilize an immunoglobulin fusion protein that does not interact with a Fc receptor and does not initiate ADCC reactions. In such instances, the immunoglobulin heterologous sequence of the fusion protein can be mutated to inhibit such reactions. See, e.g., U.S. Pat. No. 5,985,279 and WO 98/06248.

The heterologous amino acid sequence of the fusion polypeptides or conjugate fusion polypeptides utilized as part of the present invention can also comprise an amino acid sequence useful for identifying, tracking or purifying the fusion polypeptide, e.g., can comprise a FLAG or a His tag sequence. The fusion polypeptide or conjugate fusion polypeptide can further comprise an amino acid sequence containing a proteolytic cleavage site which can, for example, be useful for removing the heterologous amino acid sequence from the substance exhibiting Tubercin and/or SSM activity and/or Z-100 or a functional derivative thereof or from the a substance exhibiting Tubercin and/or SSM activity and/or Z-100 or a functional derivative thereof/conjugate fusion polypeptide.

In particular, the heterologous amino acid sequence of the fusion polypeptides or conjugate fusion polypeptides of the present invention can also comprise an amino acid sequence useful for identifying, tracking or purifying the fusion polypeptide, e.g., can comprise a FLAG (see, e.g., Hoop, T. P. et al., Bio/Technology 6, 1204-1210 (1988); Prickett, K. S. et al., BioTechniques 7, 580-589 (1989)) or a His tag (Van Reeth, T. et al., BioTechniques 25, 898-904 (1998)) sequence. The fusion polypeptide or conjugate fusion polypeptides can further comprise an amino acid sequence containing a proteolytic cleavage site which can, for example, be useful for removing the heterologous amino acid sequence from the substance exhibiting Tubercin and/or SSM activity and/or Z-100 or a functional derivative thereof.

In yet another embodiment, the fusion polypeptide or conjugate fusion polypeptide comprises a GST fusion protein in which the substance exhibiting Tubercin and/or SSM activity and/or Z-100 or a functional derivative thereof or the substance exhibiting Tubercin and/or SSM activity and/or Z-100 or a functional derivative thereof/conjugate fusion polypeptide of the invention is fused to the C-terminus of GST sequences. Such a fusion protein can facilitate the purification of a recombinant polypeptide of the invention. In those embodiments in which a GST, FLAG or HisTag fusion constructs is employed in the construction of the substance exhibiting Tubercin and/or SSM activity and/or Z-100 or a functional derivative thereof conjugate fusion proteins, proteolytic cleavage sites may be optionally introduced at the junction of the fusion moiety and the substance exhibiting Tubercin and/or SSM activity and/or Z-100 or a functional derivative thereof to enable separation of the substance exhibiting Tubercin and/or SSM activity and/or Z-100 or a functional derivative thereof from the fusion moiety subsequent to purification of the substance exhibiting Tubercin and/or SSM activity and/or Z-100 or a functional derivative thereof. Such enzymes, and their cognate recognition sequences, include, for example, without limitation, Factor Xa, thrombin and enterokinase. Typical fusion expression vectors include pGEX (Pharmacia Biotech Inc.; Smith and Johnson (1988) Gene 67:31-40), pMAL (New England Biolabs, Beverly, Mass.) and pRIT5 (Pharmacia, Piscataway, N.J.) which may be used to fuse glutathione S-transferase (GST), maltose E binding protein, or protein A, respectively, to the target substance exhibiting Tubercin and/or SSM activity or a functional derivative thereof.

Expression vectors can routinely be designed for expression of a fusion polypeptide of the invention in prokaryotic (e.g., E. coli) or eukaryotic cells (e.g., insect cells (using baculovirus expression vectors), yeast cells or mammalian cells). Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, MA) and pRIT5 (Pharmacia, Piscataway, NJ) which fuse glutathione S-transferase (GST), maltose E binding protein, or protein A, respectively, to the target recombinant protein.

Examples of suitable inducible non-fusion E. coli expression vectors include pTrc (Amann et al., (1988) Gene 69:301-315) and pET I Id (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gnl). This viral polymerase is supplied by host strains BL21 (DE3) or HMS 174(DE3) from a resident prophage harboring a T7 gnl gene under the transcriptional control of the lacUV 5 promoter.

One strategy to maximize recombinant protein expression in E. coli is to express the protein in a host bacterium with an impaired capacity to proteolytically cleave the recombinant protein (Gottesman, Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al. (1992) Nucleic Acids Res. 20:2111-2118). Such alteration of nucleic acid sequences of the invention can be carried out by standard DNA synthesis techniques.

In another embodiment, the expression vector is a yeast expression vector. Examples of vectors for expression in yeast S. cerivisae include pYepSecl (Baldari et al. (1987) EMBO J 6:229-234), pMFa (Kuijan and Herskowitz, (1982) Cell 30:933-943), pJRY88 (Schultz et al. (1987) Gene 54:113-123), pYES2 (Invitrogen Corporation, San Diego, Calif.), and pPicZ (Invitrogen Corp, San Diego, Calif.).

Alternatively, the expression vector is a baculovirus expression vector. Baculovirus vectors available for expression of proteins in cultured insect cells (e.g., Sf9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed (1987) Nature 329:840) and pMT2PC (Kaufinan et al. (1987) EMBO J 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., supra.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J 8:729-733) and immunoglobulins (Banedji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Patent No. 4,873,316 and European Application Publication No. 264,166). Developmentally-regulated promoters are also encompassed, for example the murine hox promoters (Kessel and Gruss (1990) Science 249:374-379) and the alpha-fetoprotein promoter (Campes and Tilghman (1989) Genes Dev. 3:537-546).

A host cell can be any prokaryotic (e.g., E. coli) or eukaryotic cell (e.g., insect cells, yeast or mammalian cells).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

Modes of Administration

Modes of administration of the various therapeutic agents used in the invention are exemplified below. However, the agents can be delivered by any of a variety of routes including: by injection (e.g., subcutaneous, intramuscular, intravenous, intraarterial, intraperitoneal), by continuous intravenous infusion, cutaneously, dermally, transdermally, orally (e.g., tablet, pill, liquid medicine), by implanted osmotic pumps (e.g., Alza Corp.), by suppository or aerosol spray.

The peptide conjugates used in the Tubercin and/or SSM-and/or Z-100 protein/peptide combination therapy of the present invention may be administered as free peptides or pharmaceutically acceptable salts thereof. Those skilled in the art of biochemical synthesis will recognize that for commercial-scale quantities of peptide conjugates, such peptide conjugates are preferably prepared using recombinant DNA techniques, synthetic techniques, or chemical derivatization of biologically or chemically synthesized peptides.

The peptide conjugates used in the Tubercin and/or SSM-and/or Z-100 protein/peptide combination therapy may be prepared by any suitable synthesis method such as originally described by Merrifield, J. Am. Chem. Soc., 85, p 2149 (1963). In addition, methods for the addition of saccharides to protein are known to those skilled in the art. For example, addition of sialyl Lewis acid X to antibodies for targeting purposes is described in U.S. Pat. No. 5,723,583; and modification of oligosaccharides to form vaccines is described in U.S. Pat. No. 5,370,872. A general strategy for forming protein-saccharide conjugates is outlined in U.S. Pat. No. 5,554,730. Moreover, additional synthesis methods may be found in United States Patent Application 20040214228, the entire contents of which are incorporated herein by reference in their entirety.

The terms used herein conform to those found in Budavari, Susan (Editor), “The Merck Index” An Encyclopedia of Chemicals, Drugs, and Biologicals; Merck & Co., Inc. The term “pharmaceutically acceptable salt” refers to those acid addition salts or metal complexes of the Tubercin and/or SSM and/or Z-100 compounds and or functional equivalent thereof or peptide conjugates used in the Tubercin and/or SSM and/or Z-100-protein/peptide combination therapy which do not significantly or adversely affect the therapeutic properties (e.g. efficacy, toxicity, etc.) of the compounds or peptides conjugates. The compounds or peptide conjugates should be administered to individuals as a pharmaceutical composition, which, in most cases, will comprise the compounds or peptides and/or pharmaceutical salts thereof with a pharmaceutically acceptable carrier. The term “pharmaceutically acceptable carrier” refers to those solid and liquid carriers, which do not significantly or adversely affect the therapeutic properties of the compounds or peptide conjugates.

The pharmaceutical compositions containing compounds or peptide conjugates of the present invention may be administered to individuals, particularly humans, either intravenously, subcutaneously, intramuscularly, intranasally, orally, topically, transdermally, parenterally, gastrointestinally, transbronchially and transalveolarly. Topical administration is accomplished via a topically applied cream, gel, rinse, etc. containing therapeutically effective amounts of the Tubercin and/or SSM and/or Z-100 compounds described herein and/or their functional derivatives. Transdermal administration is accomplished by application of a cream, rinse, gel, etc. capable of allowing the Tubercin and/or SSM and/or Z-100 compounds described herein and/or their functional derivatives to penetrate the skin and enter the blood stream. Parenteral routes of administration include, but are not limited to, direct injection such as intravenous, intramuscular, intraperitoneal or subcutaneous injection. Gastrointestinal routes of administration include, but are not limited to, ingestion and rectal. Transbronchial and transalveolar routes of administration include, but are not limited to, inhalation, either via the mouth or intranasally and direct injection into an airway, such as through a tracheotomy, tracheostomy, endotracheal tube, or metered dose or continuous inhaler. In addition, osmotic pumps may be used for administration. The necessary dosage will vary with the particular condition being treated, method of administration and rate of clearance of the molecule from the body.

Although the Tubercin and/or SSM and/or Z-100 compounds described herein and/or their functional derivatives may be administered as the pure chemicals, it is preferable to present the active ingredient as a pharmaceutical composition. The invention thus further provides the use of a pharmaceutical composition comprising one or more Tubercin and/or SSM and/or Z-100 compounds and/or their functional derivatives and/or a pharmaceutically acceptable salt thereof, together with one or more pharmaceutically acceptable carriers therefore and, optionally, other therapeutic and/or prophylactic ingredients. The carrier(s) must be acceptable in the sense of being compatible with the other ingredients of the composition and not deleterious to the recipient thereof.

Pharmaceutical compositions include those suitable for oral or parenteral (including intramuscular, subcutaneous, cutaneous, inhaled and intravenous) administration. The compositions may, where appropriate, be conveniently presented in discrete unit dosage forms and may be prepared by any of the methods well known in the art of pharmacy. Such methods include the step of bringing into association the active compound with liquid carriers, solid matrices, semi-solid carriers, finely divided solid carriers or combinations thereof, and then, if necessary, shaping the product into the desired delivery system.

Pharmaceutical compositions suitable for oral administration may be presented as discrete unit dosage forms such as hard or soft gelatin capsules, cachets or tablets, each containing a predetermined amount of the active ingredient; as a powder or as granules; as a solution, a suspension or as an emulsion. The active ingredient may also be presented as a bolus, electuary or paste. Tablets and capsules for oral administration may contain conventional excipients such as binding agents, fillers, lubricants, disintegrants, or wetting agents. The tablets may be coated according to methods well known in the art., e.g., with enteric coatings.

Oral liquid preparations may be in the form of, for example, aqueous or oily suspension, solutions, emulsions, syrups or elixirs, or may be presented as a dry product for constitution with water or another suitable vehicle before use. Such liquid preparations may contain conventional additives such as suspending agents, emulsifying agents, non-aqueous vehicles (which may include edible oils), or preservative. The compounds may also be formulated for parenteral administration (e.g., by injection, for example, bolus injection or continuous infusion) and may be presented in unit dose form in ampoules, pre-filled syringes, small bolus infusion containers or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g., sterile, pyrogen-free water, before use.

For topical administration to the epidermis, the compounds or peptide conjugates may be formulated as ointments, creams or lotions, or as the active ingredient of a transdermal patch. Suitable transdermal delivery systems are disclosed, for example, in Fisher et al. (U.S. Pat. No. 4,788,603) or Bawas et al. (U.S. Pat. Nos. 4,931,279, 4,668,504 and 4,713,224). Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The active ingredient can also be delivered via iontophoresis, e.g., as disclosed in U.S. Pat. Nos. 4,140,122, 4,383,529, or 4,051,842. At least two types of release are possible in these systems. Release by diffusion occurs when the matrix is non-porous. The pharmaceutically effective compound dissolves in and diffuses through the matrix itself. Release by microporous flow occurs when the pharmaceutically effective compound is transported through a liquid phase in the pores of the matrix.

Compositions suitable for topical administration in the mouth include unit dosage forms such as lozenges comprising active ingredient in a flavored base, usually sucrose and acacia or tragacanth; pastilles comprising the active ingredient in an inert base such as gelatin and glycerin or sucrose and acacia; mucoadherent gels, and mouthwashes comprising the active ingredient in a suitable liquid carrier.

When desired, the above-described compositions can be adapted to provide sustained release of the active ingredient employed, e.g., by combination thereof with certain hydrophilic polymer matrices, e.g., comprising natural gels, synthetic polymer gels or mixtures thereof.

The pharmaceutical compositions according to the invention may also contain other adjuvants such as flavorings, coloring, antimicrobial agents, or preservatives.

It will be further appreciated that the amount of the Tubercin and/or SSM and/or Z-100 and/or a functional derivative compound, or an active salt or derivative thereof, required for use in treatment will vary not only with the particular salt selected but also with the route of administration, the nature of the condition being treated and the age and condition of the patient and will be selected, ultimately, at the discretion of the attending physician.

A pharmaceutical composition of the invention contains an appropriate pharmaceutically acceptable carrier as defined supra. These compositions can take the form of solutions, suspensions, tablets, pills, capsules, powders, sustained-release formulations and the like. Suitable pharmaceutical carriers are described in Remington's Pharmaceutical Sciences 1990, pp. 1519-1675, Gennaro, A. R., ed., Mack Publishing Company, Easton, Pa. The Tubercin and/or SSM and/or Z-100 compounds described herein and/or their functional derivatives either alone or in combination with the conjugate proteins/peptides of the invention can be administered in liposomes or polymers (see, Langer, R. Nature 1998, 392, 5). Such compositions will contain an effective therapeutic amount of the active compound together with a suitable amount of carrier so as to provide the form for proper administration to the subject.

In general, the compounds of the present invention are conveniently administered in unit dosage form; for example, containing 5 to 2000 mg, conveniently 10 to 1000 mg, most conveniently, 50 to 500 mg of active ingredient per unit dosage form.

Desirable blood levels may be maintained by continuous infusion to provide about 0.01-5.0 mg/kg/hr or by intermittent infusions containing about 0.4-20 mg/kg of the active ingredient(s). Buffers, preservatives, antioxidants and the like can be incorporated as required.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations, such as multiple inhalations from an insufflator or by application of a plurality of drops into the eye.

Actual dosage levels of active ingredients in the pharmaceutical compositions of this invention may be varied so as to obtain an amount of the active compound(s) that is effective to achieve the desired therapeutic response for a particular patient, compositions, and mode of administration. The selected dosage level will depend upon the activity of the particular pharmaceutical compound or analogue thereof of the present invention, the route of administration, the severity of the condition being treated, and the condition and prior medical history of the patient being treated. However, it is within the skill of the art to start doses of the pharmaceutical compound at levels lower than required to achieve the desired therapeutic effect and to gradually increase the dosage until the desired effect is achieved.

The pharmaceutical compositions of the present invention can be used in both veterinary medicine and human therapy. The magnitude of a prophylactic or therapeutic dose of the pharmaceutical composition of the invention in the acute or chronic management of pain associated with above-mentioned diseases or indications will vary with the severity of the condition to be treated and the route of administration. The dose, and perhaps the dose frequency, will also vary according to the age, body weight, and response of the individual patient. In general, the total daily dose range of the pharmaceutical composition of this invention is generally between about 1 to about 100 mg, preferably about 1 to about 20 mg, and more preferably about 1 to about 10 mg of active compound per kilogram of body weight per day are administered to a mammalian patient. If desired, the effective daily dose may be divided into multiple doses for purposes of administration, e.g. two to four separate doses per day. Alternatively, the total daily dose range of the active ingredient of this invention should be sufficient to increase the serum concentration of the proease inhibtor by 10-100 micromolar. It is intended herein that by recitation of such specified ranges, the ranges cited also include all those dose range amounts between the recited range. For example, in the range about 1 and 100, it is intended to encompass 2 to 99, 3-98, etc, without actually reciting each specific range. The actual preferred amounts of the active ingredient will vary with each case, according to the species of mammal, the nature and severity of the particular affliction being treated, and the method of administration. It is also understood that doses within those ranges, but not explicitly stated, such as 30 mg, 50 mg, 75 mg, etc. are encompassed by the stated ranges, as are amounts slightly outside the stated range limits.

The actual preferred amounts of the active ingredient will vary with each case, according to the species of mammal, the nature and severity of the particular affliction being treated, and the method of administration. In general, the pharmaceutical compositions of the present invention are periodically administered to an individual patient as necessary to improve symptoms of the particular disease being treated. The length of time during which the compositions are administered and the total dosage will necessarily vary with each case, according to the nature and severity of the particular affliction being treated and the physical condition of the subject or patient receiving such treatment.

It is further recommended that children, patients aged over 65 years, and those with impaired renal or hepatic function initially receive low doses, and that they then be titrated based on individual response(s) or blood level(s). It may be necessary to use dosages outside these ranges in some cases, as will be apparent to those of ordinary skill in the art. Further, it is noted that the clinician or treating physician will know, with no more than routine experimentation, how and when to interrupt, adjust, or terminate therapy in conjunction with individual patient response.

Useful dosages of the compounds of the present invention can be determined by comparing their in vitro activity, and in vivo activity in animal models. Methods for the extrapolation of effective dosages in mice, and other animals, to humans are known to the art; for example, see U.S. Pat. No. 4,938,949.

### EXAMPLES

The following specific examples are provided to better assist the reader in the various aspects of practicing the present invention. As these specific examples are merely illustrative, nothing in the following descriptions should be construed as limiting the invention in any way. Such limitations are, or course, defined solely by the accompanying claims.

### Example One

A representative example is provided below of an in vivo murine S180 sarcoma bioassay to determine if one or more of the Tubercin and/or SSM and/or Z-100 and/or a finctional derivative compounds has the ability to achieve the reduction or inhibition of pain, inhibition of cancer or tumor development, inhibition of metastasis, inhibition of protein degradation, inhibition of cell proliferation in cancer cells, and/or inhibition of symptoms in any of the aforementioned methods for anyone of the aforementioned cancer diseases and/or indications.

The quantitative murine S180 sarcoma assay was performed as follows. Eight-week-old female CFW Swiss Webster mice are inoculated subcutaneously (sc) in the right flank with 4.8-4800 .mu.g/kg doses of one or more of the Tubercin and/or SSM and/or Z-100 and/or a functional derivative compounds in volume of 0.1 mL of PBS mixed with an equal volume of PBS containing 2.times.10.sup.5 viable S180 sarcoma cells. Fourteen days after injection, mice are euthanized and dissected in order to assess the incidence of tumors. Intraassay differences in the tumor incidence at 14 days (TI.sub. 14) between the control and test groups are tested for significance by Fisher's Exact Test (FET) and by the chi-squared test for the total of several assays. The bioassay may also performed by administering various doses of one or more of the Tubercin and/or SSM and/or Z-100 and/or a functional derivative compounds through several alternative routes two hours after sc S180 cell challenge, including intraperitoneal (ip) injection, ipsilateral and contralateral sc injections are made at the site of tumor challenge.

Those Tubercin and/or SSM and/or Z-100 and/or functional derivative compounds that inhibit the growth of the sarcoma cells are indentified and serve as cancer inhibiting therapies either alone or in combination with other existing anticancer agents.

### Example Two

The Effect of the Combined use of a substances exhibiting Tubercin and/or SSM activity and/or Z-100 activity or a functional derivative thereof and Cyclophosphamide.

This example demonstrates the usefulness of substances exhibiting Tubercin and/or SSM activity and/or Z-100 activity or a functional derivative thereof of the present invention in cancer therapies, particularly when the substances exhibiting Tubercin and/or SSM activity and/or Z-100 activity or a functional derivative thereof are combined with one or more of other existing anticancer agents.

Mice are administered, by a subcutaneous injection in their abdominal section, 0.2 nm of 10% Ehrlich ascites tumor cell solution (1.4×10⁷ cells) in accordance with the method of Baillif [Baillif, R. N., Caner Research, 15, 554-558(1954)], either with or.without cyclophosphamide.

A similar number of mice receive saline solution alone as control. Subsequently, the test and control mice are subjected to a subcutaneous injection of a 1 mg/ml solution of a substance exhibiting Tubercin and/or SSM activity and/or Z-100 activity or a finctional derivative thereof in 0.9% physiological saline. The injection is repeated over a period of several days. Fourteen days later after the initial inoculation with cancer cells, each of the treated mice are anesthetized.

The solid tumor growing in the abdominal section is carefully isolated from other organ tissues, cleaned using filter papers and weighed. For the test and the control group, the average weight of the cancerous tissue was tabulated and evaluated by the t-test. The results reveal that the administration of cyclophosphamide or a substance exhibiting Tubercin and/or SSM activity and/or Z-100 activity or a functional derivative thereof alone produced anticancer effects of similar magnitude, while the combined use of cyclophosphamide and a substance exhibiting Tubercin and/or SSM activity and/or Z-100 activity or a functional derivative thereof resulted in a marked decrease in the cancerous growth. This result demonstrates the usefulness of a substance exhibiting Tubercin and/or SSM activity and/or Z-100 activity or a functional derivative thereof in cancer therapies, particularly when combined with one or more of other existing anticancer agents.

### Example 3

Screening and Selection of Anti-Cancer Drugs

The present invention provides assays for identifying a substance exhibiting Tubercin and/or SSM activity and/or Z-100 activity or a functional derivative thereof that may prevent cancer.

For example, a method for identifying a compound affecting a cancer cell is provided wherein a cell is contacted with a substance exhibiting Tubercin and/or SSM activity and/or Z-100 activity or a functional derivative thereof and determination is made of whether the substance exhibiting Tubercin and/or SSM activity and/or Z-100 activity or a functional derivative thereof has the ability to achieve the reduction or inhibition of pain, inhibition of cancer or tumor development, inhibition of metastasis, inhibition of protein degradation, inhibition of cell proliferation in cancer cells, and/or inhibition of symptoms in any of the aforementioned methods for anyone of the aforementioned cancer diseases and/or indications.

By way of example, and not by way of limitation, using cell cultures of brain cancer cells, or even individual cancer cells, selection of promising drug candidates, and the evaluation of efficacy of various anti-cancer drugs for treating such cancer can be performed in the laboratory, either manually or using automated apparatus. For example, glioblastoma cells can be administered various doses of a substance exhibiting Tubercin and/or SSM activity and/or Z-100 activity or a functional derivative thereof, either alone in combination with known anti-cancer drugs, and a determination is made of whether the substance exhibiting Tubercin and/or SSM activity and/or Z-100 activity or a functional derivative thereof is capable of inhibiting the growth of the glioblastoma cells. Any changes in the growth of the glioblastoma cells, as compared with normal cells, would indicate potential for the therapeutic.

### EQUIVALENTS

Throughout this application various publications and patents are referenced. The disclosures of these publications and patents in their entireties are hereby incorporated by reference into this application in order to more fully describe the state of the art to which this invention pertains.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A method for treating a cancer in a mammal comprising administering to a subject in need thereof of a therapeutically effective amount of a composition comprising a substance exhibiting Tubercin and/or SSM activity and/or Z-100 or a functional derivative thereof; and a pharmaceutically acceptable excipient.

2. A method of relieving or ameliorating the pain or symptoms associated with one or more cancer diseases or indications in a mammal suffering from one or more cancer diseases or indications comprises administering to the mammal in need thereof a therapeutically effective pain or symptom-reducing amount of a pharmaceutical composition comprising effective amounts of a substance exhibiting Tubercin and/or SSM activity and/or Z-100 or a functional derivative thereof; and a pharmaceutically acceptable carrier or excipient, wherein said Tubercin and/or SSM activity and/or Z-100 or a functional derivative thereof substance is sufficient to relieve or ameliorate the pain or symptoms associated with one or more cancer diseases or indications.

3. A method of relieving or ameliorating cancer or tumor development, metastasis, protein degradation, cell proliferation in cancer cells, and/or inhibition of symptoms associated with cancer diseases and/or cancer indications in a mammal suffering from a cancer disease or cancer indication which comprises administering to the mammal in need thereof a therapeutically effective pain or symptom-reducing amount of a pharmaceutical composition comprising effective amounts of a substance exhibiting Tubercin and/or SSM activity and/or Z-100 activity or a functional derivative thereof and a pharmaceutically acceptable excipient, wherein said substance exhibiting Tubercin and/or SSM activity and/or Z-100 activity or a functional derivative thereof is sufficient to inhibit the cancer or tumor development, metastasis, protein degradation, and/or cell proliferation in cancer cells.

4. The method of claim 1, wherein the therapeutically effective amount of one or more substances exhibiting Tubercin and/or SSM activity and/or Z-100 or functional derivatives thereof may be administered to a subject in need thereof in conjunction with a therapeutically effective amount of one or more anti-inflammatory compounds and/or a therapeutically effective amount of one or more immunomodulatory agents.

5. The method according to claim 4, wherein the anti-inflammatory compound or immunomodulatory drug comprises interferon; interferon derivatives comprising betaseron, .beta.-interferon; prostane derivatives comprising iloprost, cicaprost; glucocorticoids comprising cortisol, prednisolone, methyl-prednisolone, dexamethasone; immunsuppressives comprising cyclosporine A, FK-506, methoxsalene, thalidomide, sulfasalazine, azathioprine, methotrexate; lipoxygenase inhibitors comprising zileutone, MK-886, WY-50295, SC-45662, SC-41661A, BI-L-357; leukotriene antagonists; peptide derivatives comprising ACTH and analogs thereof; soluble TNF-receptors; TNF-antibodies; soluble receptors of interleukines, other cytokines, T-cell-proteins; antibodies against receptors of interleukines, other cytokines, T-cell-proteins; and calcipotriols and analogues thereof taken either alone or in combination.

6. The method according to claim 1, wherein the therapeutically effective amount of one or more substances exhibiting Tubercin and/or SSM activity and/or Z-100 or a functional derivative thereof may be administered to a subject in need thereof in conjunction with one or more antimicrobial or antiviral compositions or any combination thereof.

7. The method according to claim 1, wherein the reduction or inhibition of pain and/or symptoms associated with the one or more of cancer or cancer indications is on the order of about 10-20%, 30-40% 50-60%, or 75-100% reduction or inhibition.

8. The method according to claim 3, wherein the cancer or cancer indication is selected from the group consisting of AIDS-Associated Cancers, Bladder Cancer, Bone Cancer, Brain & Spinal Cord Cancers, Metastatic Brain Tumors, Pediatric Brain Tumors, Breast Cancer, Male Breast Cancer, Cervical Cancer, Colorectal Cancer, Endometrial & Other Uterine Cancers, Esophageal Cancer, Gallbladder & Bile Duct Cancers, Gastric (Stomach) Cancer, Gestational Trophoblastic Disease, Head & Neck Cancers, Kidney Cancer, Leukemia, Liver Cancer, Liver Metastases, Lung Cancer, Lymphomas, Melanoma, Multiple Myeloma, Myelodysplastic Syndrome, Neuroblastoma, Ovarian Cancer, Pancreatic Cancer, Pediatric Cancers, Pituitary Tumors, Prostate Cancer, Rare Hematologic Disorders, Rare Solid Tumors, Retinoblastoma, Skin Cancer, Soft-Tissue Sarcoma, Testicular Cancer, Thyroid Cancer, Uterine Cancers, Wilms' Tumor, Bronchus Cancer, Colon and Rectum Cancer, Urinary Cancer, Non-Hodgkin Lymphoma, Melanomas of the Skin, Kidney and Renal Cancer, Pelvis Cancer, Pancreatic Cancer, Oral Cavity Cancer and Pharynx Cancer, Ovary Cancer, Stomach Esophagus Cancer, Intrahepatic Bile Duct Cancer, Cervix Cancer, Larynx Cancer, Acute Myeloid Leukemia, Chronic Lymphocytic Leukemia, Soft Tissue Cancer including Heart, GIST (Gastro-Intestinal Stromal Tumors), Small Intestine Cancer, Chronic Myeloid Leukemia, Acute Lymphocytic Leukemia Anus, Anal, Canal and Anorectum Cancers, Vulva Cancer, Gallbladder Cancer, Pleura Cancer, Malignant Mesothelioma, Cancer of the Bones and Joints, Hypopharynx Cancer, Eye and Orbit Cancer, Nose and Nasal Cavity Cancer, Middle Ear Cancer, Nasopharynx Cancer, Ureter Cancer, Peritoneum Cancer, Omentum and Mesentery Cancer, and Gastrointestinal Carcinoid Tumors or any combination thereof.

9. The method according to claim 7, wherein the cancer that is inhibited or prevented is selected from the group consisting of AIDS-Associated Cancers, Bladder Cancer, Bone Cancer, Brain & Spinal Cord Cancers, Metastatic Brain Tumors, Pediatric Brain Tumors, Breast Cancer, Male Breast Cancer, Cervical Cancer, Colorectal Cancer, Endometrial & Other Uterine Cancers, Esophageal Cancer, Gallbladder & Bile Duct Cancers, Gastric (Stomach) Cancer, Gestational Trophoblastic Disease, Head & Neck Cancers, Kidney Cancer, Leukemia, Liver Cancer, Liver Metastases, Lung Cancer, Lymphomas, Melanoma, Multiple Myeloma, Myelodysplastic Syndrome, Neuroblastoma, Ovarian Cancer, Pancreatic Cancer, Pediatric Cancers, Pituitary Tumors, Prostate Cancer, Rare Hematologic Disorders, Rare Solid Tumors, Retinoblastoma, Skin Cancer, Soft-Tissue Sarcoma, Testicular Cancer, Thyroid Cancer, Uterine Cancers, Wilms' Tumor, Bronchus Cancer, Colon and Rectum Cancer, Urinary Cancer, Non-Hodgkin Lymphoma, Melanomas of the Skin, Kidney and Renal Cancer, Pelvis Cancer, Pancreatic Cancer, Oral Cavity Cancer and Pharynx Cancer, Ovary Cancer, Stomach Esophagus Cancer, Intrahepatic Bile Duct Cancer, Cervix Cancer, Larynx Cancer, Acute Myeloid Leukemia, Chronic Lymphocytic Leukemia, Soft Tissue Cancer including Heart, GIST (Gastro-Intestinal Stromal Tumors), Small Intestine Cancer, Chronic Myeloid Leukemia, Acute Lymphocytic Leukemia Anus, Anal, Canal and Anorectum Cancers, Vulva Cancer, Gallbladder Cancer, Pleura Cancer, Malignant Mesothelioma, Cancer of the Bones and Joints, Hypopharynx Cancer, Eye and Orbit Cancer, Nose and Nasal Cavity Cancer, Middle Ear Cancer, Nasopharynx Cancer, Ureter Cancer, Peritoneum Cancer, Omentum and Mesentery Cancer, and Gastrointestinal Carcinoid Tumors or any combination thereof.

10. The method according to claims 1, 2 or 3, wherein the cancer disease or cancer disorder is selected from the group consisting of colorectal cancer, gastric cancer, ovarian cancer, osteosarcoma, hepatocellular carcinoma, Burkitt's lymphoma, primary effusion lymphomas, angioimmunoblastic lymphadenopathy, acquired immune deficiency syndrome (AIDS)-related lymphoma, T-cell lymphomas, oral hairy leukoplakia, lymphoproliferative disease, lymphoepithelial carcinoma, body-cavity-based lymphoma or B-cell lymphomas, non-keratinising carcinoma, squamous cell nasopharyngeal carcinoma, kidney transplant-associated epithelial tumors, angiosarcoma, Kaposi's sarcoma, angiolymphoid hyperplasia, prostatic neoplasm, retinoblastoma, Li-Fraumeni syndrome, Gardner's syndrome, Werner's syndrome, nervoid basal cell carcinoma syndrome, neurofibromatosis type 1, cervical dysplasia, neuro-blastoma, primary macroglobulinemia, insulinoma, mycosis fungoides, osteogenic sarcoma, premalignant skin lesions (topical), rhabdomyosarcoma, osteogenic, polycythemia vera, essential thrombocytosis or any combination thereof.

11. The method according to claim 1, wherein the pharmaceutical compositions are administered orally, systemically, via an implant, intravenously, topically, intrathecally, by inhalation, or nasally.

12. The method according to claim 1, wherein the substance exhibiting Tubercin and/or SSM activity and/or Z-100 or a functional derivative thereof may be part of a fusion polypeptide, wherein said fusion polypeptide comprises a substance exhibiting Tubercin and/or SSM activity and/or Z-100 or a functional derivative thereof and an amino acid sequence heterologous to said substance exhibiting Tubercin and/or SSM activity and/or Z-100 or a functional derivative thereof substance.
